SEQUENCE LISTING (2 sequences)

SEQ ID NO: 1 - DNA, 2670 bp, S. aureus
atggctgaattacctcaatcaagaataaatgaacgaaatattaccagtgaaatgcgtgaatcatttttagattatgcgatgagtgttatcgttgctcgtgcattgccagatgttcgtgacggtttaaaaccagtacatcgtcgtatactatatggattaaatgaacaaggtatgacaccggataaatcatataaaaaatcagcacgtatcgttggtgacgtaatgggtaaatatcaccctcatggtgactcatctatttatgaagcaatggtacgtatggctcaagatttcagttatcgttatccgcttgttgatggccaaggtaactttggttcaatggatggagatggcgcagcagcaatgcgttatactgaagcgcgtatgactaaaatcacacttgaactgttacgtgatattaataaagatacaatagattttatcgataactatgatggtaatgaaagagagccgtcagtcttacctgctcgattccctaacttgttagccaatggagcatcaggtatagcggtaggtatggcaacgaatattccaccacataacttaacagaattaatcaatggtgtacttagcttaagtaagaaccctgatatttcaattgctgagttaatggaggatattgaaggtcctgatttcccaactgctggacttattttaggtaagagtggtattagacgtgcatatgaaacaggtcgtggttcaattcaaatgcgttctcgtgcagttattgaagaacgtggaggcggacgtcaacgtattgttgtcactgaaattcctttccaagtgaataaggctcgtatgattgaaaaaattgcagagctcgttcgtgacaagaaaattgacggtatcactgatttacgtgatgaaacaagtttacgtactggtgtgcgtgtcgttattgatgtgcgtaaggatgcaaatgctagtgtcattttaaataacttatacaaacaaacacctcttcaaacatcatttggtgtgaatatgattgcacttgtaaatggtagaccgaagcttattaatttaaaagaagcgttggtacattatttagagcatcaaaagacagttgttagaagacgtacgcaatacaacttacgtaaagctaaagatcgtgcccacattttagaaggattacgtatcgcacttgaccatatcgatgaaattatttcaacgattcgtgagtcagatacagataaagttgcaatggaaagcttgcaacaacgcttcaaactttctgaaaaacaagctcaagctattttagacatgcgtttaagacgtctaacaggtttagagagagacaaaattgaagctgaatataatgagttattaaattatattagtgaattagaaacaatcttagctgatgaagaagtattactacaattagttagagatgaattaacagaaattcgagatcgtttcggtgatgatcgtcgtactgaaatccaattaggtggatttgaagatttagaagatgaagatctcattccagaagaacaaattgtaattacactaagccataataactacattaaacgtttgccggtatctacatatcgtgctcaaaaccgtggtggtcgtggtgttcaaggtatgaatacattggaagaagattttgtcagtcaattggtaactttaagtacacatgaccatgtattgttctttactaacaaaggtcgtgtatacaaacttaaaggttatgaagtgcctgagttatcaagacagtctaaaggtattcctgtagtgaatgctattgaacttgaaaatgatgaagtcattagtacaatgattgctgttaaagaccttgaaagtgaagacaacttcttagtgtttgcaactaaacgtggtgtcgttaaacgttcagcattaagtaacttctcaagaataaatagaaatggtaagattgcgatttcgttcagagaagatgatgagttaattgcagttcgcttaacaagtggtcaagaagatatcttgattggtacatcacatgcatcattaattcgattccctgaatcaacattacgtcctttaggccgtacagcaacgggtgtgaaaggtattacacttcgtgaaggtgacgaagttgtagggcttgatgtagctcatgcaaacagtgttgatgaagtattagtagttactgaaaatggttatggtaaacgtacgccagttaatgactatcgtttatcaaatcgtggtggtaaaggtattaaaacagctacgattactgagcgtaatggtaatgttgtatgtatcactacagtaactggtgaagaagatttaatgattgttactaatgcaggtgtcattattcgactagatgttgcagatatttctcaaaatggtcgtgcagcacaaggtgttcgcttaattcgcttaggtgatgatcaatttgtttcaacggttgctaaagtaaaagaagatgcagaagatgaaacgaatgaagatgagcaatctacttcaactgtatctgaagatggtactgaacaacaacgtgaagcggttgtaaatgatgaaacaccaggaaatgcaattcatactgaagtgattgattcagaagaaaatgatgaagatggacgtattgaagtaagacaagatttcatggatcgtgttgaagaagatatacaacaatcatcagatgaagatgaagaataa

SEQ ID NO: 2 - DNA, 1935 bp, S. aureus
atggtgactgcattgtcagatgtaaacaacacggataattatggtgctgggcaaatacaagtattagaaggtttagaagcagtacgtaaaagaccaggtatgtatataggatcgacttcagagagaggtttgcaccatttagtgtgggaaattgtcgataatagtatcgatgaagcattagctggttatgcaaataaaattgaagttgttattgaaaaagataactggattaaagtaacggataacggacgtggtatcccagttgatattcaagaaaaaatgggacgtccagctgtcgaagttattttaactgttttacatgctggtggtaaattcggcggtggcggatacaaagtatctggtggtttacatggtgttggttcatcagttgtaaacgcattgtcacaagacttagaagtatatgtacacagaaatgagactatatatcatcaagcatataaaaaaggtgtacctcaatttgacttaaaagaagttggcacaactgataagacaggtactgtcattcgttttaaagcagatggagaaatcttcacagagacaactgtatacaactatgaaacattacagcagcgtattagagagcttgctttcttaaacaaaggaattcaaatcacattaagagatgaacgtgatgaagaaaacgttagagaagactcctatcactatgagggcggtattaaatcgtacgttgagttattgaacgaaaataaagaacctattcatgatgagccaatttatattcatcaatctaaagatgatattgaagtagaaattgcgattcaatataactcaggatatgccacaaatcttttaacttacgcaaataacattcatacgtatgaaggtggtacgcatgaagacggattcaaacgtgcattaacgcgtgtcttaaatagttatggtttaagtagcaagattatgaaagaagaaaaagatagactttctggtgaagatacacgtgaaggtatgacagcaattatatctatcaaacatggtgatcctcaattcgaaggtcaaacgaagacaaaattaggtaattctgaagtgcgtcaagttgtagataaattattctcagagcactttgaacgatttttatatgaaaatccacaagtcgcacgtacagtggttgaaaaaggtattatggcggcacgtgcacgtgttgctgcgaaaaaagcgcgtgaagtaacacgtcgtaaatcagcgttagatgtagcaagtcttccaggtaaattagccgattgctctagtaaaagtcctgaagaatgtgagattttcttagtcgaaggggactctgccggggggtctacaaaatctggtcgtgactctagaacgcaggcgattttaccattacgaggtaagatattaaatgttgaaaaagcacgattagatagaattttgaataacaatgaaattcgtcaaatgatcacagcatttggtacaggaatcggtggcgactttgatctagcgaaagcaagatatcacaaaatcgtcattatgactgatgccgatgtggatggagcgcatattagaacattgttattaacattcttctatcgatttatgagaccgttaattgaagcaggctatgtgtatattgcacagccaccgttgtataaactgacacaaggtaaacaaaagtattatgtatacaatgatagggaacttgataaacttaaatctgaattgaatccaacaccaaaatggtctattgcacgatacaaaggtcttggagaaatgaatgcagatcaattatgggaaacaacaatgaaccctgagcaccgcgctcttttacaagtaaaacttgaagatgcgattgaagcggaccaaacatttgaaatgttaatgggtgacgttgtagaaaaccgtagacaatttatagaagataatgcagtttatgcaaacttagacttctaa

(19) United States
(12) Patent Application Publication
(10) Pub. No.: US 2016/0143878 A1
(43) Pub. Date: May 26, 2016

(54) ANTIBACTERIAL AGENT

(71) Applicant: JUNTENDO EDUCATIONAL FOUNDATION, TOKYO (JP)

(72) Inventors: Keiichi HIRAMATSU, Tokyo (JP); Yuh MORIMOTO, Tokyo (JP); Tadashi BABA, Tokyo (JP)

(73) Assignee: JUNTENDO EDUCATIONAL FOUNDATION, TOKYO (JP)

(21) Appl. No.: 15/017,932

(22) Filed: Feb. 8, 2016

Related U.S. Application Data
(63) Continuation of application No. 14/441,973, filed on May 11, 2015.

(30) Foreign Application Priority Data
Nov. 13, 2012 (JP) 2012-249415

(51) Int. Cl. A61K 31/352; C12N 9/90; A61K 45/06

(52) U.S. Cl. CPC A61K 31/352; A61K 45/06; C12N 9/90; C12Y 599/01003

(57) ABSTRACT

An antibacterial agent comprising a compound represented by the following general formula (I), which can exhibit potent antibacterial activity against bacteria that have acquired resistance to quinolones (in the formula, R¹ and R⁴ represents hydrogen atom, hydroxyl group, or a lower alkoxy group; R² and R³ represents hydrogen atom or hydroxyl group; R⁵ represents hydrogen atom, a lower cyclic alkyl group, or a lower alkoxy group; R^{1′} and R^{5′} represents hydrogen atom; R^{2′} and R^{3′} represents hydrogen atom, hydroxyl group, or a lower alkoxy group; and R^{4′} represents hydrogen atom or hydroxyl group).

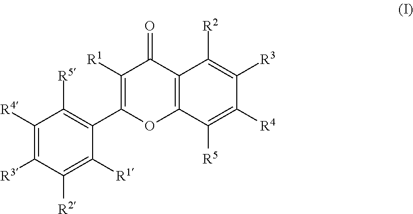

### CROSS-REFERENCE TO RELATED APPLICATIONS

This application is a Continuation of U.S. application Ser. No. 14/441,973, which is the National Stage of International Application No. PCT/JP2013/080486, filed Nov. 12, 2013, which claims priority to Japanese Patent Application No. 2012-249415, filed Nov. 13, 2012. The disclosure of application Ser. No. 14/441,973 and PCT/JP2013/080486 are expressly incorporated by reference herein in their entireties.

### TECHNICAL FIELD

The present invention relates to an antibacterial agent comprising a flavone compound as an active ingredient. More specifically, the present invention relates to an antibacterial agent that exhibits a potent antibacterial activity against microorganisms that have acquired resistance to quinolones.

### BACKGROUND ART

Quinolones are synthetic antibacterial agents that inhibit the activity of DNA gyrase to inhibit religation of cleaved DNA strands, thereby causing the DNA replication failure, and eventually inhibit DNA synthesis of bacteria. They have been widely used as drugs or animal drugs.

As compounds having an action similar to that of quinolones, several flavones have been reported, and those flavones have an inhibitory action against the DNA gyrase of Escherichia coli (see, Non-patent documents 1 and 2). However, in the aforementioned reports, it has been demonstrated that the minimum inhibitory concentrations (MIC) of the aforementioned flavones for Staphylococcus aureus are 62.5 mg/L or higher, and thus they do not show antibacterial activity against Staphylococcus aureus.

As for Staphylococcus aureus, there are continuously increasing multiply antibiotic-resistant strains, of which typical example is the methicillin-resistant Staphylococcus aureus (MRSA) that acquired resistance to β-lactam antibacterial agents. It is no exaggeration to say that there is no hospital where MRSA does not exist, and it poses a serious clinical problem.

As an only antibacterial agent that has long maintained its effectiveness to multiply antibiotic-resistant MRSA, vancomycin is available. However, the inventors of the present invention found vancomycin-intermediate S. aureus (VISA) in 1996 to which vancomycin therapy was ineffective (see, Non-patent document 3). Since then, VISA has been found in various countries in the world so far to date. Moreover, vancomycin-resistant S. aureus (VRSA), which is MRSA highly resistant to vancomycin, was found in the U.S. in 2003, and at present, 9 strains in total have been reported from three countries; i.e., the U.S., Iran, and India (see, Non-patent document 4).

Mu50, which is a typical strain of VISA (see, Non-patent document 3), is resistant to, for example, quinolone, β-lactam, tetracycline, minocycline, aminoglycoside, macrolide, rifampicin, glycopeptide, and the like. It is also reported that almost all the VISA strains separated in various countries in the world exhibit similar multiple resistance.

Therefore, development of new antibacterial agents that show superior antibacterial activity against multiply antibiotic-resistant bacteria is strongly desired, and also as for quinolones, development of new antibacterial agents that show superior antibacterial activity against quinolone-resistant Staphylococcus aureus including the aforementioned MRSA, VISA, and VRSA as well as other quinolone-resistant Gram-positive bacteria is strongly desired.

It is understood that the resistance against quinolones is acquired through introduction of a mutation at a specific position of gyrase as the target enzyme of quinolones, which reduces the gyrase inhibitory action of quinolones. As an antibacterial agent effective to such a mutated gyrase, nybomycin is known (Non-patent document 5). Although the antibacterial activity of nybomycin against quinolone-susceptible bacteria is weak, nybomycin shows notably potent antibacterial activity against bacteria that have acquired resistance to quinolones (resistant bacteria having a gyrase including a mutation such as Ser84Leu) (Non-patent document 5). Further, it has also been reported that reversion occurs in gyrase in bacteria, that have acquired resistance to nybomycin, and they become susceptible to quinolones (Non-patent document 5).

If an antibacterial agent that has the property as mentioned above can be newly provided, it can be used in combination with a quinolone so that bacteria that have acquired resistance to the quinolone are effectively eliminated by the antibacterial agent, and at the same time, bacteria that have acquired resistance to the antibacterial agent can be eliminated with the quinolone, and therefore it is expected that an extremely effective treatment of infectious diseases can be achieved with preventing emergence of new resistant bacteria.

### PRIOR ART REFERENCES

### Non-Patent Documents

Non-patent document 1: Ohemeng K. A., Podlogar B. L., Nguyen V. N., Bernstein J. I., Krause H. M., Hilliard J. J., Barrett J. F., J. Med. Chem., 1997 Sep. 26; 40(20), pp. 3292-3296
Non-patent document 2: Hilliard J. J., Krause H. M., Bernstein J. I., Fernandez J. A., Nguyen V., Ohemeng K. A., Barrett J. F., Adv. Exp. Med. Biol., 1995, 390, pp. 59-69
Non-patent document 3: Hiramatsu, K., Hanaki H., et al., J. Antimicrob. Chemother., 1997, 40(1), 135-136
Non-patent document 4: Perichon B., Courvalin P., Antimicrob. Agents Chemother., 2009, November; 53(11), 4580-7, Epub 2009 Jun. 8
Non-patent document 5: Hiramatsu K. et al., Int. J. Antimicrob. Agents, 2012, 39(6), pp. 478-485

### SUMMARY OF THE INVENTION

### Object to be Achieved by the Invention

An object of the present invention is to provide an antibacterial agent that has a potent antibacterial activity against bacteria with acquired resistance to quinolones. More specifically, the object of the present invention is to provide a novel antibacterial agent that has a potent antibacterial activity against bacteria with acquired resistance to quinolones through introduction of a mutation into gyrase. Another object of the present invention is to provide an antibacterial agent that can prevent emergence of resistant bacteria when it is used in combination with a quinolone.

### Means for Achieving the Object

In order to achieve the aforementioned objects, the inventors of the present invention conducted extensive studies. As a result, they found that flavone compounds represented by the following general formula (I) had a potent antibacterial activity against bacteria that had acquired resistance to quinolones, and that they had a potent antibacterial activity against bacteria that had acquired resistance to quinolones through introduction of a mutation into gyrase. They also found that by combining the compounds with a quinolone, an antibacterial agent that can prevent emergence of resistant bacteria is successfully provided. The present invention was accomplished on the basis of these findings.

The present invention thus provides an antibacterial agent for use in a prophylactic and/or therapeutic treatment of an infectious disease caused by a bacterium that has acquired resistance to quinolones, which comprises, as an active ingredient, a compound represented by the following general formula (I):

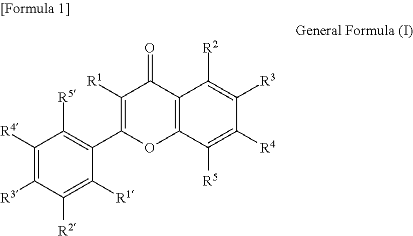

wherein, R¹ represents hydrogen atom, hydroxyl group, or a lower alkoxy group; R² represents hydrogen atom or hydroxyl group; R³ represents hydrogen atom or hydroxyl group; R⁴ represents hydrogen atom, hydroxyl group, or a lower alkoxy group; R⁵ represents hydrogen atom, a lower cyclic alkyl group, or a lower alkoxy group; R^{1′} represents hydrogen atom; R^{2′} represents hydrogen atom, hydroxyl group, or a lower alkoxy group; R^{3′} represents hydrogen atom, hydroxyl group, or a lower alkoxy group; R^{4′} represents hydrogen atom or hydroxyl group; and R^{5′} represents hydrogen atom.

According to a preferred embodiment of the present invention, there is provided the aforementioned antibacterial agent, wherein R¹ is hydrogen atom, hydroxyl group, or methoxy group, R⁴ is hydrogen atom, hydroxyl group, or methoxy group, W is hydrogen atom, cyclopropyl group, or methoxy group, R^{2′} is hydrogen atom, hydroxyl group, or methoxy group, and R^{3′} is hydrogen atom, hydroxyl group, or methoxy group.

As other aspects of the present invention, there are provided an inhibitor against a DNA gyrase of a quinolone-resistant bacterium, which comprises a compound represented by the aforementioned general formula (I) as an active ingredient, and a therapeutic agent for an infectious disease caused by a quinolone-resistant bacterium, which comprises a compound represented by the aforementioned general formula (I) as an active ingredient.

There are also provided a therapeutic agent for an infectious disease, which is used for suppressing emergence and/or proliferation of a quinolone-resistant bacterium, and comprises a compound represented by the aforementioned general formula (I) as an active ingredient, and a therapeutic agent for an infectious disease, which is used in a therapeutic treatment of an infectious disease using a quinolone for suppressing emergence and/or proliferation of a quinolone-resistant bacterium, and comprises a compound represented by the aforementioned general formula (I) as an active ingredient.

As a further aspect of the present invention, there is provided a medicament for a prophylactic and/or therapeutic treatment of an infectious disease, which comprises a combination of the aforementioned antibacterial agent and a quinolone. This medicament can be used as a medicament for suppressing emergence and/or proliferation of a quinolone-resistant bacterium. The aforementioned medicament may be a medicament comprising a combination of the aforementioned antibacterial agent and the quinolone as separate unit dosage forms, or may be a medicament that comprises the aforementioned antibacterial agent and the quinolone in a single dosage form.

There are also provided use of a compound represented by the aforementioned general formula (I) for manufacture of the aforementioned antibacterial agent; a method for a prophylactic and/or therapeutic treatment of an infectious disease of a mammal including human caused by a bacterium that has acquired resistance to quinolones, which comprises the step of administrating a prophylactically and/or therapeutically effective amount of a compound represented by the aforementioned general formula (I) to the mammal; and the aforementioned method, which comprises the step of administrating a prophylactically and/or therapeutically effective amount of a quinolone to the mammal simultaneously with or separately from the compound represented by the aforementioned general formula (I).

### Effect of the Invention

According to the present invention, there is provided an antibacterial agent that can show potent antibacterial activity against quinolone-resistant bacteria. The action of the antibacterial agent of the present invention is similar to the action of nybomycin, and the agent can exhibit notably potent antibacterial activity against bacteria that have acquired resistance to quinolones (resistant bacteria containing a gyrase having a mutation such as Ser84Leu), although the antibacterial activity thereof against bacteria susceptible to quinolones is weak. Further, in a bacterium that has acquired resistance to the antibacterial agent of the present invention, reverse mutation occurs in the gyrase, and thus it becomes susceptible to quinolones. Therefore, with a combination of the antibacterial agent of the present invention and a quinolone, bacteria that have acquired resistance to quinolones are effectively eliminated with the antibacterial agent of the present invention, and at the same time, bacteria that have acquired resistance to the antibacterial agent of the present invention can be eliminated with the quinolone, and thus an extremely effective treatment of infectious diseases can be achieved by the prevention of emergence of new resistant bacteria. Accordingly, the medicament of the present invention comprising a combination of the antibacterial agent of the present invention and a quinolone is useful for a prophylactic and/or therapeutic treatment of an infectious disease caused by either one or both of a quinolone-susceptible bacterium and a quinolone-resistant bacterium, and it can attain high prophylactic and/or therapeutic effect by preventing emergence of quinolone-resistant bacteria.

### BRIEF DESCRIPTION OF THE DRAWINGS

FIG. 1 shows the results of the DNA cleavage assay performed with GyrA(wt) and GyrB(wt) in the absence or presence of levofloxacin (LVFX). “S” represents a substrate plasmid DNA having a superhelix structure, “L” represents linear double-stranded DNA formed by cleavage of the aforementioned substrate plasmid DNA with the DNA gyrase, and “N” represents double-stranded DNA in which one strand of the double-stranded substrate plasmid DNA was cleaved by the DNA gyrase.

FIG. 2 shows the results of the DNA cleavage assay performed with GyrA(wt) and GyrB(wt) in the absence or presence of apigenin. “S”, “L”, and “N” have the same meanings as those explained for FIG. 1.

FIG. 3 shows the results of the DNA cleavage assay performed with GyrA(S84L) and GyrB(wt) in the absence or presence of levofloxacin (LVFX). “S”, “L”, and “N” have the same meanings as those explained for FIG. 1.

FIG. 4 shows the results of the DNA cleavage assay performed with GyrA(S84L) and GyrB(wt) in the absence or presence of apigenin. “S”, “L”, and “N” have the same meanings as those explained for FIG. 1.

### MODES FOR CARRYING OUT THE INVENTION

In this specification, the term “lower” means a carbon number of about 1 to 6, preferably a carbon number of 1 to 4.

Examples of the lower cyclic alkyl group include, for example, cyclopropyl group, cyclobutyl group, cyclopentyl group, cyclohexyl group, and the like, and cyclopropyl group is preferred. On the ring of the lower cyclic alkyl group, one or two or more of lower alkyl groups such as methyl group, lower alkoxy groups such as methoxy group, hydroxyl groups, or halogen atoms such as fluorine atom may substitute.

Examples of the lower alkoxy group include methoxy group, ethoxy group, propoxy group, and the like, and methoxy group is preferred. The lower alkoxy group may have a substituent. Type, number, and substituting position of the substituent are not particularly limited, and when there are two or more substituents, they may be the same or different. Examples of the substituent of the lower alkoxy group include, for example, a halogen atom, hydroxyl group, cyano group, and the like, but it is not limited to these.

When the compounds represented by the general formula (I) form a salt, a physiologically acceptable salt may be used as the active ingredient of the antibacterial agent of the present invention. Further, a hydrate or solvate of the compounds represented by the general formula (I) or a salt thereof can also be used as the active ingredient. Although type of the solvent that forms the solvate is not particularly limited, examples include, for example, ethanol, ethyl acetate, acetone, and the like. The compounds represented by general formula (I) may exist as an enantiomer, diastereoisomer, or geometrical isomer depending on type of the substituent. An arbitrary isomer in pure form or a mixture of arbitrary isomers can be used as the active ingredient of the antibacterial agent of the present invention.

Preferred examples of the compounds represented by the general formula (I) include 3,3′,4′,5,7-pentahydroxyflavone hydrate (compound 1 mentioned in Table 11, the compound wherein R¹ is hydroxyl group, R² is hydroxyl group, R³ is hydrogen atom, R⁴ is hydroxyl group, R⁵ is hydrogen atom, R^{1′} is hydrogen atom, R^{2′} is hydroxyl group, R^{3′} is hydroxyl group, R^{4′} is hydrogen atom, and R^{5′} is hydrogen atom, also referred to as quercetin dihydrate), 3,4′,5,7-tetrahydroxyflavone hydrate (compound 4 mentioned in Table 11, the compound wherein R¹ is hydroxyl group, R² is hydroxyl group, R³ is hydrogen atom, R⁴ is hydroxyl group, R⁵ is hydrogen atom, R^{1′} is hydrogen atom, R^{2′} is hydrogen atom, R^{3′} is hydroxyl group, R^{4′} is hydrogen atom, and R^{5′} is hydrogen atom, also referred to as kaempferol hydrate), 3,5,7-trihydroxy-2-(4-hydroxy-3-methoxyphenyl)-8-methoxychromen-4-one (compound 38 mentioned in Table 11, the compound wherein R¹ is hydroxyl group, R² is hydroxyl group, R³ is hydrogen atom, R⁴ is hydroxyl group, R⁵ is methoxy group, R^{1′} is hydrogen atom, R^{2′} is methoxy group, R^{3′} is hydroxyl group, R^{4′} is hydrogen atom, and R^{5′} is hydrogen atom, also referred to as limocitrin), 3′,4′,5,7-tetrahydroxyflavone (compound 6 mentioned in Table 11, the compound wherein R¹ is hydrogen atom, R² is hydroxyl group, R³ is hydrogen atom, R⁴ is hydroxyl group, R⁵ is hydrogen atom, R^{1′} is hydrogen atom, R^{2′} is hydroxyl group, R^{3′} is hydroxyl group, R^{4′} is hydrogen atom, and R^{5′} is hydrogen atom, also referred to as luteolin), 4′,5,7-trihydroxyflavone (compound 7 mentioned in Table 11, the compound wherein R¹ is hydrogen atom, R² is hydroxyl group, R³ is hydrogen atom, R⁴ is hydroxyl group, R⁵ is hydrogen atom, R^{1′} is hydrogen atom, R^{2′} is hydrogen atom, is hydroxyl group, R^{4′} is hydrogen atom, and R^{5′} is hydrogen atom, also referred to as apigenin), 7-methoxyflavone (compound 15 mentioned in Table 11, the compound wherein R¹ is hydrogen atom, R² is hydrogen atom, R³ is hydrogen atom, R⁴ is methoxy group, R⁵ is hydrogen atom, R^{1′} is hydrogen atom, R^{2′} is hydrogen atom, R^{3′} is hydrogen atom, R^{4′} is hydrogen atom, and R^{5′} is hydrogen atom), 2-(4-hydroxyphenyl)-7-methoxychromen-4-one (compound 69 mentioned in Table 11, the compound wherein R¹ is hydrogen atom, R² is hydrogen atom, R³ is hydrogen atom, R⁴ is methoxy group, W is hydrogen atom, IV is hydrogen atom, R^{2′} is hydrogen atom, R^{3′} is hydroxyl group, R^{4′} is hydrogen atom, and R^{5′} is hydrogen atom, also referred to as isopratol), 5-hydroxy-2-(3-hydroxy-4,5-dimethoxyphenyl)-3,7-dimethoxychromen-4-one (compound 24 mentioned in Table 11, the compound wherein R¹ is methoxy group, R² is hydroxyl group, R³ is hydrogen atom, R⁴ is methoxy group, R⁵ is hydrogen atom, R^{1′} is hydrogen atom, R^{2′} is methoxy group, R^{3′} is methoxy group, R^{4′} is hydroxyl group, and W′ is hydrogen atom, also referred to as myricetin 3,7,3′,4′-tetramethyl ether), 5-hydroxy-2-(4-hydroxy-3-methoxyphenyl)-3,7-dimethoxychromen-4-one (compound 66 mentioned in Table 11, the compound wherein R¹ is methoxy group, R² is hydroxyl group, R³ is hydrogen atom, R⁴ is methoxy group, R⁵ is hydrogen atom, R^{1′} is hydrogen atom, R^{2′} is methoxy group, R^{3′} is hydroxyl group, R^{4′} is hydrogen atom, and R^{5′} is hydrogen atom, also referred to as pachypodol), and the like.

Preferred examples of the compounds represented by the general formula (I) also include the compound wherein R¹ is hydrogen atom, R² is hydrogen atom, R³ is hydrogen atom, R⁴ is methoxy group, R⁵ is hydrogen atom, R^{1′} is hydrogen atom, R^{2′} is methoxy group, R^{3′} is hydroxyl group, R^{4′} is hydrogen atom, and R^{5′} is hydrogen atom (M-246), the compound wherein R¹ is hydroxyl group, R² is hydroxyl group, R³ is hydroxyl group, R⁴ is hydroxyl group, R⁵ is hydrogen atom, R^{1′} is hydrogen atom, R^{2′} is hydrogen atom, R^{3′} is hydrogen atom, R^{4′} is hydrogen atom, and R^{5′} is hydrogen atom (M-21), the compound wherein R¹ is hydrogen atom, R² is hydrogen atom, R³ is hydrogen atom, R⁴ is hydroxyl group, R⁵ is cyclopropyl group, R^{1′} is hydrogen atom, R^{2′} is hydrogen atom, R^{3′} is hydroxyl group, R^{4′} is hydrogen atom, and R^{5′} is hydrogen atom (J-Q21), and the compound wherein R¹ is hydrogen atom, R² is hydroxyl group, R³ is hydrogen atom, R⁴ is hydroxyl group, R⁵ is cyclopropyl group, R^{1′} is hydrogen atom, R^{2′} is hydrogen atom, R^{3′} is hydroxyl group, R^{4′} is hydrogen atom, and R^{5′} is hydrogen atom (J-Q12.1). M-246 and M-21 can be easily obtained as commercial products, and J-Q21 and J-Q12.1 can be easily synthesized by the methods specifically described in the section of Examples in this specification. However, the compounds represented by the general formula (I) are not limited to these preferred compounds.

Among these, in view of degree of the inhibitory activity for the DNA gyrase of Staphylococcus aureus, apigenin, myricetin 3,7,3′,4′-tetramethyl ether, isopratol, luteolin, kaempferol hydrate, M-246, M-21, J-Q21, and J-Q12.1 are preferred, apigenin, myricetin 3,7,3′,4′-tetramethyl ether, isopratol, M-246, and J-Q21 are more preferred, and apigenin, M-246, and J-Q21 are particularly preferred.

The antibacterial agent of the present invention has an action of inhibiting DNA gyrase of bacteria. The DNA gyrase is an essential factor for the DNA replication having a function of duly advancing DNA polymerase when the DNA polymerase advances along a template strand during DNA replication by repeatedly cleaving and religating DNA strands to ease the superhelix structure which is inevitably produced when the DNA polymerase advances along a template strand and makes extension of the replication difficult. The antibacterial agent of the present invention has a potent inhibitory activity against a mutated gyrase of, in particular, a bacterium that has acquired resistance to quinolones, preferably a Gram-positive bacterium that has acquired resistance to quinolones, particularly preferably Staphylococcus aureus that has acquired resistance to quinolones.

For example, Staphylococcus aureus that has acquired resistance to quinolones has a mutant Staphylococcus aureus DNA gyrase A subunit in which a leucine residue substitutes for the 84th serine residue from the amino terminus of the wild-type DNA gyrase A subunit of Staphylococcus aureus, and the antibacterial agent of the present invention can preferably inhibit the activity of such mutant Staphylococcus aureus DNA gyrase A subunit. Therefore, the antibacterial agent of the present invention can exhibit a potent antibacterial activity specifically against a bacterium that has acquired resistance to quinolones.

Many of the compounds represented by the general formula (I) are marketed, and they can be easily obtained. They can also be easily synthesized by known methods or methods similar to the known methods, or they can also be extracted from natural products. For example, apigenin is an ingredient contained in known foodstuffs such as celery and parsley, and therefore it is preferred because of high safety thereof.

As the antibacterial agent of the present invention, a compound represented by the general formula (I) which is the active ingredient, per se, may be used. It is generally desirable to provide the agent in the form of a pharmaceutical composition by using pharmaceutical additives. A single kind of pharmaceutical additive may be used, or two or more kinds of pharmaceutical additives may be used in combination.

Pharmaceutical additives can be appropriately chosen depending on a purpose of use, and examples include, for example, antibacterial agents, preservatives, caking agents, thickening agents, sticking agents, binders, colorants, stabilizers, pH adjustors, buffering agents, isotonic agents, solvents, anti-oxidants, ultraviolet inhibitors, crystal precipitation inhibitors, antifoams, physicochemical property improvers, antiseptics, and the like.

Dosage form of the antibacterial agent of the present invention, preferably the antibacterial agent in the form of a pharmaceutical composition, is not particularly limited, and examples include, for example, solid agents, semi-solid agents, liquid agents, and the like. Examples of the solid agent include tablets, chewable tablets, effervescent tablets, orally disintegrating tablets, troches, drops, hard capsules, soft capsules, granules, powders, pills, dry syrups, infusions, and the like. When the agent is used as an external preparation, the dosage form may be, for example, suppositories, cataplasms, plasters, or the like. Examples of the semi-solid agent include, for example, jellies, ointments, creams, mousses, and the like. Examples of the dosage form of the liquid agent suitable for oral administration include, for example, syrups, suspensions, and the like, and examples of the dosage form of the liquid agent suitable for parenteral administration include, for example, injections for intravenous administration, intramuscular administration, or subcutaneous administration, drip infusions, eye drops, ear drops, nasal drops, aerosols, nebulas, and the like.

A method for administration, a dose, a time for administration, and an object of administration of the antibacterial agent of the present invention are not particularly limited, and they can be appropriately chosen depending on a purpose of use. The dose can be appropriately chosen in consideration of various factors such as the age, body weight, sex, symptoms of an individual as the target of administration, and whether other medicaments are administered or not. Examples of species of the object of the administration include, for example, human, apes, swines, bovines, ovines, caprines, canines, felines, mouse, rat, birds, and the like, but is not limited to these examples.

In the antibacterial agent of the present invention, a single kind of the compound represented by the general formula (I) may be used as the active ingredient, or two or more kinds of the compounds may be used in combination. Furthermore, the agent may be used in combination with other medicinal active ingredients.

The antibacterial agent of the present invention can exhibit an especially potent antibacterial activity against bacteria that have acquired resistance to quinolones. The antibacterial agent of the present invention can exhibit a potent antibacterial activity against both Gram-positive bacteria and Gram-negative bacteria that have acquired resistance to quinolones through introduction of a mutation into DNA gyrase. The agent exhibits especially superior antibacterial activity against, in particular, Gram-positive bacteria, preferably Staphylococcus aureus, that have acquired resistance to quinolones. Therefore, by combining the antibacterial agent of the present invention with a quinolone, a potent antibacterial activity against both quinolone-susceptible bacteria and quinolone-resistant bacteria can be exhibited.

It is known that, in a bacterium such as Staphylococcus aureus that has acquired resistance to quinolones, amino acid mutations are introduced into the DNA gyrase (topoisomerase II) and top oisomerase IV. The aforementioned DNA gyrase is encoded by the gyrA and gyrB genes, and the aforementioned topoisomerase IV is encoded by parCand parE. Examples of the quinolone-resistant Staphylococcus aureus include, for example, methicillin-resistant Staphylococcus aureus (MRSA), vancomycin-intermediate resistant Staphylococcus aureus (VISA), vancomycin-resistant Staphylococcus aureus (VRSA), and the like. The antibacterial agent of the present invention has an antibacterial activity especially suitable for, for example, Staphylococcus aureus having a mutant DNA gyrase A subunit in which a leucine residue substitutes for the 84th serine residue from the amino terminus of the DNA gyrase A subunit of wild-type Staphylococcus aureus.

Although the method for measuring the antibacterial activity is not particularly limited, there can generally be used the method of determining the minimum inhibitory concentration (henceforth also referred to as “MIC”) and the like, and the broth microdilution method and the like are preferably used.

MIC of the antibacterial agent of the present invention for quinolone-resistant bacteria such as quinolone-resistant Staphylococcus aureus is preferably, for example, 32 mg/L or lower, more preferably 16 mg/L or lower, still more preferably 8 mg/L or lower, particularly preferably 4 mg/L or lower. If MIC is higher than 4 mg/L, the antibacterial activity is rather weak, and proliferation of a bacterium may not be sufficiently inhibited.

The antibacterial agent of the present invention has a potent antibacterial activity against bacteria that have acquired resistance to quinolones. However, if a quinolone-resistant bacterium further acquires resistance to the antibacterial agent of the present invention, the mutation introduced into the DNA gyrase is reversed, and the DNA gyrase becomes a non-mutated DNA gyrase of a quinolone-susceptible bacterium. Therefore, by using a quinolone and the antibacterial agent of the present invention in combination, emergence of bacteria resistant to quinolones can be effectively prevented.

Quinolones are generally classified into the quinolones of the first generation (old quinolones), and the quinolones of the second generation (new quinolones). Specific examples of the old quinolones include nalidixic acid, piromidic acid, pipemidic acid, and the like. Since the new quinolones exhibit high antibacterial activity against not only Gram-negative bacteria, but also Gram-positive bacteria, they are also widely used for infectious diseases induced by pneumococci or streptococci, besides urogenital infectious diseases, enteric infections, and the like. Specific examples of the new quinolones include norfloxacin, levofloxacin, ofloxacin, enoxacin, ciprofloxacin chloride, tosufloxacin tosylate, lomefloxacin hydrochloride, fleroxacin, sparfloxacin, gatifloxacin, puzufloxacin mesylate, and the like, but not limited to these examples. When the antibacterial agent of the present invention and a quinolone are used in combination, the quinolone may consist of a single kind of quinolone or two or more kinds of quinolones. A combination with a new quinolone is preferred.

When the antibacterial agent of the present invention and at least one kind of quinolone are used in combination, the antibacterial agent of the present invention and at least one kind of the quinolone may be administered in combination as separate agents (separate dosage forms), or as a single agent containing the both in a unit dosage form (so-called a combination drug). Times of the administration of the antibacterial agent of the present invention and the quinolone are not particularly limited, and can be appropriately chosen depending on a purpose of use. The antibacterial agent of the present invention and at least one kind of quinolone may be simultaneously administered, or they may be successively administered with an appropriately chosen time interval so long as the effect of the present invention is not degraded. When the antibacterial agent of the present invention and at least one kind of quinolone are administered with a time interval, order of the administration of the antibacterial agent of the present invention and the quinolone is not particularly limited, and can be appropriately determined depending on a purpose of use.

The antibacterial agent of the present invention can be used for a prophylactic and/or therapeutic treatment of infectious diseases caused by various bacteria. In particular, use of the antibacterial agent of the present invention in combination with at least one kind of quinolone is preferred, since such use can be applied for any of an infectious disease induced by a quinolone-resistant bacterium, an infectious disease caused by a quinolone-susceptible bacterium, and an infectious disease caused by the both. Since the antibacterial agent of the present invention acts on quinolone-resistant bacteria, and the quinolone acts on quinolone-susceptible bacteria, such use is advantageous in that antibacterial activity can be obtained irrespective of the presence or absence of quinolone resistance in causative bacteria of infectious diseases, and can be widely used for a treatment of infectious diseases.

Furthermore, when a bacterium that has acquired resistance to quinolones because of mutation of gyrase further acquires resistance to the antibacterial agent of the present invention, reversion arises in the mutated gyrase, and the bacterium again becomes susceptible to quinolones. Therefore, by using the antibacterial agent of the present invention and a quinolone in combination, quinolone-susceptible bacteria can be eliminated with the quinolone, and quinolone-resistant bacteria can be effectively eliminated with the antibacterial agent of the present invention. Therefore, an extremely effective treatment of an infectious disease can be attained with preventing emergence of new quinolone-resistant bacteria.

### EXAMPLES

The present invention will be specifically explained with reference to the following examples. However, the scope of the present invention is not limited by these examples.

### Example 1

Antibacterial activities of 133 kinds of the compounds shown in Tables 1 to 10 mentioned below against Staphylococcus aureus were examined by the following methods.

### Preparation of Test Compounds

The compounds shown in Tables 1 to 10 mentioned below (flavones, isoflavones, flavanones, anthocyanidins, and flavans) were each dissolved in dimethyl sulfoxide (DMSO) at a concentration of 1,024 mg/L. Cation-adjusted MHB was separately prepared by adding magnesium and calcium to MHB (Mueller Hinton Broth, produced by Becton Dickinson) according to CLSI (Clinical and Laboratory Standards Institute) (henceforth also referred to as “CAMHB”), and each solution of test compound was diluted 4 times with CAMHB to prepare a solution of a concentration of 256 mg/L. This solution was serially 2-fold-diluted 11 times in total to a concentration of 0.125 mg/L.

<Measurement of MIC of Test Compound for Staphylococcus aureus>

### —Preparation of Cell Suspension—

S. aureus Mu50 (refer to Hiramatsu, K., Hanaki H., et al., J. Antimicrob. Chemother., 1997, 40(1), 135-6) or S. aureus FDA209P (ATCC 6538P) was cultured overnight at 37° C. in 4 mL of TBS (Tryptical Soy Broth, produced by Becton Dickinson) with shaking. After completion of the culture, the cells were suspended in fresh TBS to prepare a cell suspension showing an absorbance of 0.3 at 578 nm. Then, the cell suspension was diluted 500 times with CAMHB mentioned above.

### —Measurement of MIC (Minimum Inhibitory Concentration)—

CAMHB containing each test compound at each of the aforementioned concentrations was added to each well of a 96-well plate in a volume of 50 μL/well, the aforementioned cell suspension diluted 500 time was added to the well in a volume of 50 μL/well, and stationary culture was carried out overnight at 37° C. The final concentrations of the test compounds at the time of culture were 128 to 0.0625 mg/L.

After completion of the culture, whether the bacterium proliferated or not was determined on the basis of visually observed turbidity of the culture in the well, and MIC values (mg/L) of the test compounds against the strains were calculated. The results are shown in Tables 1 to 10 mentioned below. It was determined that the compounds that showed an MIC value not higher than 32 mg/L had antibacterial activity.

TABLE 1
Flavones

| | | | | MIC (mg/L) | |
|---|---|---|---|---|---|
| No. | Compound | Manufacturer | Molecular formula | Mu50 | FDA209P |
| 1 | 3,3′,4′,5,7-Pentahydroxyflavone | Tokyo Chemical Industry | C₁₅H₁₀O₇•xH₂O | 32 | 64 |
| | Hydrate | | | | |
| 2 | 2′,3,4′,5,7-Pentahydroxyflavone | Tokyo Chemical Industry | C₁₆H₁₀O₇•xH₂O | 128 | >128 |
| | Hydrate | | | | |
| 3 | 2-(2,4-dihydroxyphenyl)-3,5,7- | Labotest | C₁₅H₁₀O₇ | >128 | >128 |
| | trihydroxychromen-4-one | | | | |
| 4 | 3,4′,5,7-Tetrahydroxyflavone Hydrate | Tokyo Chemical Industry | C₁₆H₁₀O₆•xH₂O | 8 | 16 |
| 5 | 2-(3,4-dihydroxyphenyl)-3,5,7- | AnalytiCon Discovery | C₁₆H₁₂O₇ | 128 | 128 |
| | trihydroxy-6-methylchromen-4-one | | | | |
| 6 | 3′,4′,5,7-Tetrahydroxyflavone | Tokyo Chemical Industry | C₁₅H₁₀O₆ | 8 | 32 |
| 7 | 4′,5,7-Trihydroxyflavone | Tokyo Chemical Industry | C₁₅H₁₀O₅ | 4 | >128 |
| 8 | 5,7-dihydroxy-8-methyl-2- | SPECS | C₁₆H₁₂O₄ | >128 | >128 |
| | phenylchromen-4-one | | | | |
| 9 | 7-acetyloxy-2-(3,4- | Pharmeks | C₂₃H₁₈O₁₁ | 64 | 128 |
| | diacetyloxyphenyl)-5-hydroxy-4- | | | | |
| | oxochromen-3-yl acetate | | | | |
| 10 | 3′,4′,5,6,7,8-Hexamethoxyflavone | Tokyo Chemical Industry | C₂₁H₂₂O₈ | 128 | 128 |
| 11 | 4′,5,6,7,8-Pentamethoxyflavone | Tokyo Chemical Industry | C₂₀H₂₀O₇ | >128 | >128 |
| 12 | 3-Hydroxyflavone | Tokyo Chemical Industry | C₁₅H₁₀O₃ | >128 | >128 |
| 13 | 6-Hydroxyflavone | Tokyo Chemical Industry | C₁₅H₁₀O₃ | >128 | >128 |
| 14 | 7,8-Dihydroxyflavone | Tokyo Chemical Industry | C₁₅H₁₀O₄ | 64 | 64 |
| 15 | 7-Methoxyflavone | Tokyo Chemical Industry | C₁₆H₁₂O₃ | 32 | 128 |
| 16 | 6-Methoxyflavone | Tokyo Chemical Industry | C₁₆H₁₂O₃ | 128 | 128 |
| 17 | 5-Hydroxyflavone | Tokyo Chemical Industry | C₁₅H₁₀O₃ | 128 | 128 |
| 18 | 5,7-dimethoxy-2-(4- | MAYBRIDGE | C₁₈H₁₆O₅ | 128 | 128 |
| | methoxyphenyl)chromen-4-one | | | | |
| 19 | 2-(3,4-dimethoxyphenyl)-3,5,7- | Pharmeks | C₂₀H₂₀O₇ | >128 | >128 |
| | trimethoxychromen-4-one | | | | |
| 20 | 5,7-dihydroxy-6-methoxy-2-(4- | Pharmeks | C₁₇H₁₄O₆ | >128 | >128 |
| | methoxyphenyl)chromen-4-one | | | | |
| 21 | 5,7-dimethoxy-8-methyl-2- | SPECS | C₁₈H₁₆O₄ | >128 | >128 |
| | phenylchromen-4-one | | | | |
| 22 | 2-(3,4-dimethoxyphenyl)-5,7- | Labotest | C₁₉H₁₈O₈ | >128 | >128 |
| | dimethoxychromen-4-one | | | | |
| 23 | 5,7-bis(methylethoxy)-8-iodo-2-[4- | SPECS | C₂₄H₂₇IO₅ | >128 | >128 |
| | (methylethoxy)phenyl]chromen-4-one | | | | |
| 24 | 5-hydroxy-2-(3-hydroxy-4,5- | AnalytiCon Discovery | C₁₉H₁₈O₈ | 8 | 128 |
| | dimethoxyphenyl)-3,7- | | | | |
| | dimethoxychromen-4-one | | | | |
| 25 | 2-phenylchromen-4-one (2-phenyl-1- | Tokyo Chemical Industry | C₁₅H₁₀O₂ | 64 | 128 |
| | benzopyran-4-one) | | | | |

TABLE 2

| | | | | MIC (mg/L) | |
|---|---|---|---|---|---|
| No. | Compound | Manufacturer | Molecular formula | Mu50 | FDA209P |
| 26 | 5,7-dihydroxy-2-(4-hydroxyphenyl)-6- | AnalytiCon Discovery | C₁₆H₁₂O₆ | >128 | >128 |
| | methoxychromen-4-one | | | | |
| 27 | 5,7-dihydroxy-2-(4-hydroxyphenyl)- | AnalytiCon Discovery | C₁₇H₁₄O₇ | >128 | >128 |
| | 3,6-dimethoxychromen-4-one | | | | |
| 28 | 5-hydroxy-2-(4-hydroxyphenyl)-6,7- | AnalytiCon Discovery | C₁₇H₁₄O₆ | >128 | >128 |
| | dimethoxychromen-4-one | | | | |
| 29 | 5,7-dihydroxy-2-(4-hydroxy-3- | AnalytiCon Discovery | C₁₈H₁₆O₈ | >128 | >128 |
| | methoxyphenyl)-3,6- | | | | |
| | dimethoxychromen-4-one | | | | |
| 30 | 5,6,7,8-tetramethoxy-2-(4- | AnalytiCon Discovery | C₂₀H₂O₇ | >128 | 128 |
| | methoxyphenyl)chromen-4-one | | | | |
| 31 | 2-(3,4-dihydroxyphenyl)-5-hydroxy- | AnalytiCon Discovery | C₁₇H₁₄O₇ | >128 | >128 |
| | 6,7-dimethoxychromen-4-one | | | | |
| 32 | 7,9-dimethoxy-6-(4-methoxyphenyl)- | AnalytiCon Discovery | C₁₉H₁₆O₇ | >128 | >128 |
| | 2H-1,3-dioxoleno[4,5-g]chromen-8- | | | | |
| | one | | | | |
| 33 | 5-hydroxy-2-(3-hydroxy-4- | AnalytiCon Discovery | C₁₉H₁₈O₈ | 128 | >128 |
| | methoxyphenyl)-3,6,7- | | | | |
| | trimethoxychromen-4-one | | | | |
| 34 | 5,6,7-trihydroxy-2-(4- | AnalytiCon Discovery | C₁₆H₁₂O₆ | 128 | 128 |
| | methoxyphenyl)chromen-4-one | | | | |
| 35 | 3,5,6,7,8-pentamethoxy-2-(7- | AnalytiCon Discovery | C₂₂H₂₂O₁₀ | >128 | >128 |
| | methoxy(2H-benzo[3,4-d]1,3- | | | | |
| | dioxolan-5-yl))chromen-4-one | | | | |
| 36 | 7-acetyloxy-2-(3,4- | TimTec | C₂₃H₁₈O₁₀ | >128 | >128 |
| | diacetyloxyphenyl)-4-oxochromen-5- | | | | |
| | yl acetate | | | | |
| 37 | 2-(3,4-dihydroxyphenyl)-3,5,7,8- | TimTec | C₁₅H₁₀O₈ | 64 | 128 |
| | tetrahydroxychromen-4-one | | | | |
| 38 | 3,5,7-trihydroxy-2-(4-hydroxy-3- | TimTec | C₁₇H₁₄O₈ | 32 | 128 |
| | methoxyphenyl)-8-methoxychromen- | | | | |
| | 4-one | | | | |
| 39 | 2-(3-hydroxy-4-methoxyphenyl)- | TimTec | C₁₉H₁₈O₇ | 128 | 128 |
| | 5,6,7-trimethoxychromen-4-one | | | | |
| 40 | 5-hydroxy-2-(3-hydroxy-4- | TimTec | C₁₈H₁₆O₇ | 128 | 128 |
| | methoxyphenyl)-6,7- | | | | |
| | dimethoxychromen-4-one | | | | |

TABLE 3

| | | | | MIC (mg/L) | |
|---|---|---|---|---|---|
| No. | Compound | Manufacturer | Molecular formula | Mu50 | FDA209P |
| 41 | 5,6,7-trihydroxy-2-(4- | TimTec | C₁₅H₁₀O₆ | 128 | 128 |
| | hydroxyphenyl)chromen-4-one | | | | |
| 42 | 2-(3,4-dimethoxyphenyl)-5,6,7- | TimTec | C₂₀H₂₀O₇ | >128 | >128 |
| | trimethoxychromen-4-one | | | | |
| 43 | 5,7-dimethoxy-2-(3,4,5- | TimTec | C₂₀H₂₀O₇ | >128 | >128 |
| | trimethoxyphenyl)chromen-4-one | | | | |
| 44 | 5-hydroxy-8-(4-hydroxyphenyl)-2,2- | AnalytiCon Discovery | C₂₀H₁₆O₅ | >128 | >128 |
| | dimethyl-2H-pyrano[3,2-g]chromen-6- | | | | |
| | one | | | | |
| 45 | 8-[5-(5,7-dihydroxy-4-oxochromen-2- | App Tec | C₃₀H₁₈O₁₀ | >128 | >128 |
| | yl)-2-hydroxyphenyl]-5,7-dihydroxy-2- | | | | |
| | (4-hydroxyphenyl)chromen-4-one | | | | |
| 46 | 8-(5,5-dimethyl-4-oxofuran-3-yl)-5,7- | SPECS | C₂₃H₂₀O₆ | >128 | 128 |
| | dimethoxy-2-phenylchromen-4-one | | | | |
| 47 | 8-[(dimethylamino)methyl]-7-hydroxy- | Vitas-M | C₁₉H₁₉NO₄ | >128 | 128 |
| | 5-methoxy-2-phenylchromen-4-one | | | | |
| 48 | 7-hydroxy-5-methoxy-2-phenyl-8- | Vitas-M | C₂₂H₂₃NO₄ | >128 | 128 |
| | (piperidylmethyl)chromen-4-one | | | | |
| 49 | 8-[(3,5-dimethylpiperidyl)methyl]-7- | Vitas-M | C₂₄H₂₇NO₄ | >128 | 128 |
| | hydroxy-5-methoxy-2- | | | | |
| | phenylchromen-4-one | | | | |
| 50 | 7-hydroxy-5-methoxy-8-(morpholin-4- | Pharmeks | C₂₁H₂₁NO₅ | >128 | >128 |
| | ylmethyl)-2-phenylchromen-4-one | | | | |
| 51 | 7-hydroxy-5-methoxy-8-[(4- | Pharmeks | C₂₂H₂₄N₂O₄ | 128 | 128 |
| | methylpiperazinyl)methyl]-2- | | | | |
| | phenylchromen-4-one | | | | |
| 52 | 7-hydroxy-5-methoxy-8-[(2- | Pharmeks | C₂₃H₂₅NO₄ | >128 | >128 |
| | methylpiperidyl)methyl]-2- | | | | |
| | phenylchromen-4-one | | | | |
| 53 | 7-hydroxy-5-methoxy-8-[(3- | Pharmeks | C₂₃H₂₅NO₄ | >128 | 128 |
| | methylpiperidyl)methyl]-2- | | | | |
| | phenylchromen-4-one | | | | |
| 54 | 3-Hydroxyflavone-2′-sulfonic Acid | Tokyo Chemical Industry | C₁₅H₉NAO₆SxH₂O | 128 | 128 |
| | Sodium Salt Hydrate | | | | |
| 55 | 2-piperidin-1-ylethyl 3-methyl-4-oxo- | Tokyo Chemical Industry | C₂₄H₂₅NO₄HCl | >128 | 128 |
| | 2-phenylchromene-8-carboxylate | | | | |
| 56 | 3-acetyl-2,4-dioxo-1-phenyl-1,3- | Labotest | C₁₇H₁₃NO₃ | >128 | >128 |
| | dihydroquinoline | | | | |
| 57 | 5,6-Benzoflavone | Tokyo Chemical Industry | C₁₉H₁₂O₂ | 128 | 128 |
| 58 | 7,8-Benzoflavone | Tokyo Chemical Industry | C₁₉H₁₂O₂ | 128 | 128 |
| 59 | 5,6-Dihydroxy-4-oxo-2-phenyl-4H-1- | Tokyo Chemical Industry | C₂₁H₁₈O₁₁ | 128 | 128 |
| | benzopyran-7-yl β-D- | | | | |
| | Glucopyranosiduronic Acid | | | | |
| 60 | 8-[(3R,4R,5R,6R)-3,4,5-trihydroxy-6- | AnalytiCon Discovery | C₂₁H₂₀O₁₁ | >128 | >128 |
| | (hydroxymethyl)(2H-3,4,5,6- | | | | |
| | tetrahydropyran-2-yl)]-2-(3,4- | | | | |
| | dihydroxyphenyl)-5,7- | | | | |
| | dihydroxychromen-4-one | | | | |

TABLE 4

| | | | | MIC (mg/L) | |
|---|---|---|---|---|---|
| No. | Compound | Manufacturer | Molecular formula | Mu50 | FDA209P |
| 61 | 8-[(2S,4S,5S,3R,6R)-3,4,5- | TimTec | C₂₁H₂₀O₁₃ | 128 | 128 |
| | trihydroxy-6-(hydroxymethyl)(2H- | | | | |
| | 3,4,5,6-tetrahydropyran-2-yloxy)]-2- | | | | |
| | (3,4-dihydroxyphenyl)-3,5,7- | | | | |
| | trihydroxychromen-4-one | | | | |
| 62 | (2S,3S,4S,6S,5R)-6-[2-(3,4- | AnalytiCon Discovery | C₂₁H₁₈O₁₂ | >128 | >128 |
| | dihydroxyphenyl)-5-hydroxy-4- | | | | |
| | oxochromen-7-yloxy]-3,4,5- | | | | |
| | trihydroxy-2H-3,4,5,6- | | | | |
| | tetrahydropyran-2-carboxylic acid | | | | |
| 63 | 2-{4-[(2S,4S,5S,3R,6R)-3,4,5- | AnalytiCon Discovery | C₂₁H₂₀O₁₀ | 128 | 128 |
| | trihydroxy-6-(hydroxymethyl)(2H- | | | | |
| | 3,4,5,6-tetrahydropyran-2- | | | | |
| | yloxy)]phenyl}-5,7-dihydroxychromen- | | | | |
| | 4-one | | | | |
| 64 | 5,7-dihydroxy-2-(3-hydroxy-4- | AnalytiCon Discovery | C₂₁H₂₀O₁₁ | >128 | >128 |
| | {[(2S,3R,4S,5S,6R)-3,4,5-trihydroxy- | | | | |
| | 6-(hydroxymethyl)oxan-2- | | | | |
| | yl]oxy}phenyl)-4H-chromen-4-one | | | | |
| 65 | 3-[(2S,4S,5S,3R,6R)-3,4,5- | AnalytiCon Discovery | C₂₁H₂₀O₁₁ | >128 | >128 |
| | trihydroxy-6-(hydroxymethyl)(2H- | | | | |
| | 3,4,5,6-tetrahydropyran-2-yloxy)]-5,7- | | | | |
| | dihydroxy-2-(4- | | | | |
| | hydroxyphenyl)chromen-4-one | | | | |
| 66 | 5-hydroxy-2-(4-hydroxy-3- | AnalytiCon Discovery | C₁₈H₁₆O₇ | 32 | 128 |
| | methoxyphenyl)-3,7- | | | | |
| | dimethoxychromen-4-one | | | | |
| 67 | (2S,3S,4S,6S,5R)-6-[4-(5,7- | AnalytiCon Discovery | C₂₁H₁₈O₁₂ | 128 | 128 |
| | dihydroxy-4-oxochromen-2-yl)-2- | | | | |
| | hydroxyphenoxy]-3,4,5-trihydroxy-2H- | | | | |
| | 3,4,5,6-tetrahydropyran-2-carboxylic | | | | |
| | acid | | | | |
| 68 | 2-(4-hydroxyphenyl)chromen-4-one | TimTec | C₁₅H₁₀O₃ | 128 | 128 |
| 69 | 2-(4-hydroxyphenyl)-7- | TimTec | C₁₆H₁₂O₄ | 16 | >128 |
| | methoxychromen-4-one | | | | |
| 70 | 5-hydroxy-2-(4- | TimTec | C₁₅H₁₀O₄ | 128 | 128 |
| | hydroxyphenyl)chromen-4-one | | | | |

TABLE 5
Isoflavones

| | | | | MIC (mg/L) | |
|---|---|---|---|---|---|
| No. | Compound | Manufacturer | Molecular formula | Mu50 | FDA209P |
| 71 | ethyl 5,7-dihydroxy-3-(4- | Enamine | C₁₉H₁₆O₇ | 128 | 128 |
| | methoxyphenyl)-4-oxochromene-2- | | | | |
| | carboxylate | | | | |
| 72 | 7-acetyloxy-3-(4-methoxyphenyl)-4- | Enamine | C₂₀H₁₆O₇ | >128 | >128 |
| | oxochromen-5-yl acetate | | | | |
| 73 | 3-(3,4-dimethoxyphenyl)-5,7- | Enamine | C₁₈H₁₆O₆ | >128 | 128 |
| | dihydroxy-2-methylchromen-4-one | | | | |
| 74 | 5-hydroxy-7-methoxy-3-(4- | Labotest | C₁₇H₁₄O₅ | >128 | >128 |
| | methoxyphenyl)chromen-4-one | | | | |
| 75 | 5,7-diethoxy-3-(4- | Labotest | C₂₁H₂₂O₅ | >128 | >128 |
| | ethoxyphenyl)chromen-4-one | | | | |
| 76 | 5,7-dimethoxy-3-(4- | Labotest | C₁₈H₁₆O₅ | >128 | >128 |
| | methoxyphenyl)chromen-4-one | | | | |
| 77 | 3-(2H,3H-benzo[3,4-e]1,4-dioxan-6- | Life Chemicals | C₂₂H₁₈O₈ | >128 | >128 |
| | yl)-7-acetyloxy-2-methyl-4- | | | | |
| | oxochromen-5-yl acetate | | | | |
| 78 | 3-{4-[(2S,4S,5S,3R,6R)-3,4,5- | Life Chemicals | C₂₂H₂₂O₁₀ | >128 | >128 |
| | trihydroxy-6-(hydroxymethyl)(2H- | | | | |
| | 3,4,5,6-tetrahydropyran-2- | | | | |
| | yloxy)]phenyl}-5-hydroxy-7- | | | | |
| | methoxychromen-4-one | | | | |
| 79 | [3-(3,4-dimethoxyphenyl)-5-hydroxy- | Life Chemicals | C₂₀H₁₉NO₇ | >128 | >128 |
| | 4-oxochromen-7-yloxy]-N,N- | | | | |
| | dimethylcarboxamide | | | | |
| 80 | [3-(3,4-dimethoxyphenyl)-5-methoxy- | Life Chemicals | C₂₁H₂₁NO₇ | >128 | >128 |
| | 4-oxochromen-7-yloxy]-N,N- | | | | |
| | dimethylcarboxamide | | | | |
| 81 | 3-{4-[(4S,5S,3R,6R)-3,4,5-trihydroxy- | MAYBRIDGE | C₂₁H₂₀O₁₀ | >128 | >128 |
| | 6-(hydroxymethyl)(2H-3,4,5,6- | | | | |
| | tetrahydropyran-2-yloxy)]phenyl}-5,7- | | | | |
| | dihydroxychromen-4-one | | | | |
| 82 | 7-acetyloxy-3-(4-acetyloxyphenyl)-4- | MAYBRIDGE | C₂₁H₁₆O₈ | >128 | >128 |
| | oxochromen-5-yl acetate | | | | |
| 83 | 3-(2H,3H,4H-benzo[b]1,4-dioxepin-7- | OTAVA | C₁₉H₁₆O₆ | 128 | 128 |
| | yl)-5,7-dihydroxy-2-methylchromen-4- | | | | |
| | one | | | | |
| 84 | 5-hydroxy-3-(4-hydroxyphenyl)-7- | OTAVA | C₂₃H₂₆O₆ | >128 | >128 |
| | octyloxychromen-4-one | | | | |
| 85 | 3-{4-[(2S,4S,5S,3R,6R)-3,4,5- | Princeton | C₂₃H₂₄O₁₀ | >128 | >128 |
| | trihydroxy-6-(hydroxymethyl)(2H- | | | | |
| | 3,4,5,6-tetrahydropyran-2- | | | | |
| | yloxy)]phenyl}-5,7- | | | | |
| | dimethoxychromen-4-one | | | | |
| 86 | 5-hydroxy-7-methoxy-2-methyl-3-(7- | Princeton | C₂₀H₁₈O₆ | >128 | >128 |
| | methyl(2H,3H-benzo[3,4-e]1,4- | | | | |
| | dioxan-6-yl))chromen-4-one | | | | |
| 87 | 5,7-dihydroxy-2-methyl-3-(7- | Princeton | C₁₉H₁₆O₆ | 128 | 64 |
| | methyl(2H,3H-benzo[3,4-e]1,4- | | | | |
| | dioxan-6-yl))chromen-4-one | | | | |

TABLE 6

| | | | | MIC (mg/L) | |
|---|---|---|---|---|---|
| No. | Compound | Manufacturer | Molecular formula | Mu50 | FDA209P |
| 88 | 5,7-dimethoxy-2-methyl-3-(7- | Princeton | C₂₁H₂₀O₆ | >128 | 128 |
| | methyl(2H,3H-benzo[3,4-e]1,4- | | | | |
| | dioxan-6-yl))chromen-4-one | | | | |
| 89 | 7-acetyloxy-2-methyl-3-(7- | Princeton | C₂₃H₂₀O₈ | 128 | 128 |
| | methyl(2H,3H-benzo[3,4-e]1,4- | | | | |
| | dioxan-6-yl))-4-oxochromen-5-yl | | | | |
| | acetate | | | | |
| 90 | 2-ethyl-5,7-dihydroxy-3-(4- | SALOR | C₁₈H₁₆O₅ | >128 | 128 |
| | methoxyphenyl)chromen-4-one | | | | |
| 91 | ethyl 5-hydroxy-7-methoxy-3-(4- | SPECS | C₂₀H₁₈O₇ | >128 | >128 |
| | methoxyphenyl)-4-oxochromene-2- | | | | |
| | carboxylate | | | | |
| 92 | 5,7-dimethoxy-3-(4-methoxyphenyl)- | SPECS | C₁₉H₁₆O₇ | >128 | >128 |
| | 4-oxochromene-2-carboxylic acid | | | | |
| 93 | 5,7-dihydroxy-3-(3-hydroxy-4,5- | AnalytiCon | C₁₈H₁₆O₈ | 128 | 128 |
| | dimethoxyphenyl)-6- | | | | |
| | methoxychromen-4-one | | | | |
| 94 | 5,7-dihydroxy-6-methoxy-3-(4- | AnalytiCon | C₁₇H₁₄O₆ | 128 | 128 |
| | methoxyphenyl)chromen-4-one | | | | |
| 95 | (2S,3S,4S,6S,5R)-6-[4-(5,7- | AnalytiCon | C₂₁H₁₈O₁₁ | >128 | 128 |
| | dihydroxy-4-oxochromen-3- | | | | |
| | yl)phenoxy]-3,4,5-trihydroxy-2H- | | | | |
| | 3,4,5,6-tetrahydropyran-2-carboxylic | | | | |
| | acid | | | | |
| 96 | 5-[(4S,5S,3R,6R)-3,4,5-trihydroxy-6- | AnalytiCon | C₂₂H₂₂O₁₀ | 128 | 128 |
| | (hydroxymethyl)(2H-3,4,5,6- | | | | |
| | tetrahydropyran-2-yl)oxy]-3-(4- | | | | |
| | hydroxyphenyl)-7-methoxychromen- | | | | |
| | 4-one | | | | |
| 97 | 7-(4-hydroxyphenyl)-9-methoxy-2H- | AnalytiCon | C₁₇H₁₂O₆ | 128 | 128 |
| | 1,3-dioxoleno[4,5-g]chromen-8-one | | | | |
| 98 | 5,7-dihydroxy-3-(2,4,5- | AnalytiCon | C₁₈H₁₆O₇ | >128 | 128 |
| | trimethoxyphenyl)chromen-4-one | | | | |
| 99 | 5,7-dihydroxy-3-(4-hydroxyphenyl)-8- | AnalytiCon | C₂₁H₂₀O₁₀ | >128 | 128 |
| | [3,4,5-trihydroxy-6- | | | | |
| | (hydroxymethyl)(2H-3,4,5,6- | | | | |
| | tetrahydropyran-2-yl)]chromen-4-one | | | | |
| 100 | 5,7-dihydroxy-3-(4-hydroxyphenyl)-6- | AnalytiCon | C₁₆H₁₂O₆ | 128 | 128 |
| | methoxychromen-4-one | | | | |
| 101 | 3-(2,4-dihydroxyphenyl)-5,7- | AnalytiCon | C₁₅H₁₀O₆ | 128 | 128 |
| | dihydroxychromen-4-one | | | | |
| 102 | 2-(3-(2H,3H-benzo[3,4-e]1,4-dioxin- | Pharmeks | C₁₉H₁₄O₈ | 128 | 128 |
| | 6-yl)-5-hydroxy-4-oxochromen-7- | | | | |
| | yloxy)acetic acid | | | | |
| 103 | 3-(2H,3H,4H-benzo[b]1,4-dioxepin-7- | Pharmeks | C₂₂H₂₂O₁₀ | 128 | 128 |
| | yl)-7-acetyloxy-4-oxochromen-5-yl | | | | |
| | acetate | | | | |

TABLE 7

| | | | | MIC (mg/L) | |
|---|---|---|---|---|---|
| No. | Compound | Manufacturer | Molecular formula | Mu50 | FDA209P |
| 104 | 3-(2H,3H-benzo[3,4-e]1,4-dioxin-6- | Pharmeks | C₂₁H₁₆O₈ | 64 | 128 |
| | yl)-7-acetyloxy-4-oxochromen-5-yl | | | | |
| | acetate | | | | |
| 105 | 11-(4-hydroxyphenyl)-2,2,6,6- | Pharmeks | C₂₅H₂₄O₆ | 128 | 128 |
| | tetramethyl-6H-pyrano[2,3- | | | | |
| | h]pyrano[6,5-f]chroman-12-one | | | | |
| 106 | 3-(2H,3H-benzo[3,4-e]1,4-dioxan-6- | Pharmeks | C₁₇H₁₂O₆ | 64 | 64 |
| | yl)-5,7-dihydroxychromen-4-one | | | | |
| 107 | 3-(2H,3H,4H-benzo[b]1,4-dioxepin-7- | Pharmeks | C₁₈H₁₄O₆ | 128 | 128 |
| | yl)-5,7-dihydroxychromen-4-one | | | | |
| 108 | 3-(3,4-dimethoxyphenyl)-7-hydroxy-5- | Pharmeks | C₁₈H₁₆O₆ | 128 | 128 |
| | methoxychromen-4-one | | | | |
| 109 | 3-(2H,3H,4H-benzo[b]1,4-dioxepin-7- | Pharmeks | C₂₃H₂₀O₈ | 128 | 128 |
| | yl)-7-acetyloxy-2-methyl-4- | | | | |
| | oxochromen-5-yl acetate | | | | |
| 110 | 5-hydroxy-3-(4-methoxyphenyl)-7- | TimTec | C₂₂H₂₂O₁₀ | 128 | 128 |
| | [3,4,5-trihydroxy-6- | | | | |
| | (hydroxymethyl)(2H-3,4,5,6- | | | | |
| | tetrahydropyran-2-yloxy)]chromen-4- | | | | |
| | one | | | | |
| 111 | 3-(3,4-dimethoxyphenyl)-5,7- | TimTec | C₁₉H₁₈O₆ | 128 | >128 |
| | dimethoxychromen-4-one | | | | |
| 112 | 5-hydroxy-3-(4-hydroxyphenyl)-7- | TimTec | C₁₆H₁₂O₅ | >128 | >128 |
| | methoxychromen-4-one | | | | |
| 113 | 5,7-dihydroxy-3-(4- | Vitas-M | C₁₅H₁₀O₅ | 128 | 128 |
| | hydroxyphenyl)chromen-4-one | | | | |
| 114 | ethyl 3-(3,4-dimethoxyphenyl)-5,7- | Vitas-M | C₂₀H₁₈O₈ | >128 | >128 |
| | dihydroxy-4-oxochromene-2- | | | | |
| | carboxylate | | | | |
| 115 | 5,7-dihydroxy-3-(4-methoxyphenyl)-2- | Vitas-M | C₁₇H₁₄O₅ | 64 | 128 |
| | methylchromen-4-one | | | | |
| 116 | 5,7-dihydroxy-3-(4- | Vitas-M | C₁₆H₁₂O₅ | 128 | 128 |
| | methoxyphenyl)chromen-4-one | | | | |
| 117 | 3-(3,4-dimethoxyphenyl)-5,7- | Vitas-M | C₁₇H₁₄O₆ | >128 | >128 |
| | dihydroxychromen-4-one | | | | |
| 118 | 3-(3,4-dimethoxyphenyl)-5,7- | Vitas-M | C₁₈H₁₃F₃O₆ | 64 | 128 |
| | dihydroxy-2-(trifluoromethyl)chromen- | | | | |
| | 4-one | | | | |
| 119 | 7-acetyloxy-3-(4-methoxyphenyl)-2- | Vitas-M | C₂₁H₁₈O₇ | >128 | >128 |
| | methyl-4-oxochromen-5-yl acetate | | | | |
| 120 | 3-{4-[(2S,4S,5S,3R,6R)-3,4,5- | Vitas-M | C₂₁H₂₀O₁₀ | >128 | >128 |
| | trihydroxy-6-(hydroxymethyl)(2H- | | | | |
| | 3,4,5,6-tetrahydropyran-2- | | | | |
| | yloxy)]phenyl}-5,7-dihydroxychromen- | | | | |
| | 4-one | | | | |
| 121 | 3-(4-hydroxyphenyl)-5,7- | Vitas-M | C₁₇H₁₄O₅ | >128 | >128 |
| | dimethoxychromen-4-one | | | | |
| 122 | 5-hydroxy-2-(hydroxymethyl)-7-(4- | App Tec | C₂₀H₁₆O₆ | >128 | >128 |
| | hydroxyphenyl)-2-methyl-2H- | | | | |
| | pyrano[3,2-g]chromen-6-one | | | | |

TABLE 8
Flavanones

| | | | | MIC (mg/L) | |
|---|---|---|---|---|---|
| No. | Compound | Manufacturer | Molecular formula | Mu50 | FDA209P |
| 123 | 4′-Hydroxyflavanone | Tokyo Chemical Industry | C₁₅H₁₂O₃ | 128 | 128 |
| 124 | 3′-Hydroxyflavanone | Tokyo Chemical Industry | C₁₅H₁₂O₃ | 128 | 128 |
| 125 | 3′,5,7-Trihydroxy-4′- | Tokyo Chemical Industry | C₁₆H₁₄O₆ | >128 | 128 |
| | methoxyflavanone | | | | |
| 126 | 2,3-Dihydro-2-phenyl-4H- | Tokyo Chemical Industry | C₁₅H₁₂O₂ | 128 | 128 |
| | benzopyran-4-one | | | | |
| 127 | 7-Hydroxyflavanone | Tokyo Chemical Industry | C₁₅H₁₂O₃ | 128 | 128 |
| 128 | 4′,5,7-Trihydroxyflavanone | Tokyo Chemical Industry | C₁₅H₁₂O₆ | 128 | 128 |

TABLE 9
Anthocyanidins

| | | | | MIC (mg/L) | |
|---|---|---|---|---|---|
| No. | Compound | Manufacturer | Molecular formula | Mu50 | FDA209P |
| 129 | 2-(4-hydroxy-3- | App Tec | C₁₆H₁₃O₆ | 128 | 128 |
| | methoxyphenyl)benzo[b]oxin-3,5,7- | | | | |
| | triol | | | | |
| 130 | 2-(4-hydroxy-3,5- | App Tec | C₁₇H₁₅O₇ | 128 | 128 |
| | dimethoxyphenyl)benzo[b]oxin-3,5,7- | | | | |
| | triol | | | | |
| 131 | 2-(3,4-dihydroxy-5- | App Tec | C₁₆H₁₃O₇ | 128 | 128 |
| | methoxyphenyl)benzo[b]oxin-3,5,7- | | | | |
| | triol | | | | |

TABLE 10
Flavans

| | | | | MIC (mg/L) | |
|---|---|---|---|---|---|
| No. | Compound | Manufacturer | Molecular formula | Mu50 | FDA209P |
| 132 | (+)-3,3′,4′,5,7-Flavanpentol Hydrate | Tokyo Chemical Industry | C₁₅H₁₄O₈•xH₂O | 128 | 128 |
| 133 | 3,3′,4′,5,7-Flavanpentol Hydrate | Tokyo Chemical Industry | C₁₅H₁₄O₈•xH₂O | >128 | 128 |

As seen from the results shown in Tables 1 to 4, among 70 kinds of the flavones, 9 flavones (Nos. 1, 6, 8, 10, 26, 35, 53, 86 and 90) exhibited superior antibacterial activity against S. aureus Mu50 with MIC values of not higher than 32 mg/L. These 9 kinds of flavones had more potent antibacterial activity against quinolone-resistant methicillin-resistant Staphylococcus aureus (MRSA) Mu50 compared with the quinolone-susceptible Staphylococcus aureus FDA209P. The structures of these 9 kinds of flavones are shown in Table 11 below. The MIC value of the flavone having the fundamental structure of the test compounds (No. 37) against S. aureus Mu50 was 62 mg/L which was lower than the MIC value of the same against S. aureus FDA209P.

As seen from the results shown in Tables 5 to 7, all of 52 kinds of the isoflavones gave MIC values not lower than 64 mg/L, and thus no isoflavones were found to have antibacterial activity against S. aureus Mu50 and S. aureus FDA209P.

As seen from the results shown in Tables 8 to 10, all of 6 kinds of the flavanones, 3 kinds of the anthocyanidins, and 2 kinds of flavans gave MIC values not lower than 128 mg/L, and thus no flavanones were found to have antibacterial activity against S. aureus Mu50 and S. aureus FDA209P.

### Example 2

Antibacterial activities of 9 kinds of the flavones that showed antibacterial activity against S. aureus Mu50 in Example 1 (Nos. 1, 6, 8, 10, 26, 35, 53, 86 and 90), as well as the flavone proved to have no antibacterial activity against S. aureus Mu50 (No. 37) and 2 kinds of quinolones (levofloxacin and norfloxacin) as controls were examined according to the following methods.

### <Preparation of Flavones and Quinolones>

Ten kinds of the flavones shown in Table 11 mentioned below (Nos. 1, 6, 8, 10, 26, 35, 37, 53, 86 and 90) were each dissolved in DMSO at a concentration of 1,024 mg/L. Levofloxacin (LVFX, produced by LKT laboratories) and norfloxacin (NFLX, produced by SIGMA-ALDRICH), both are quinolones, were each dissolved in sterilized water at a concentration of 1,024 mg/L. Subsequently, these solutions of flavones and quinolones were diluted 4 times with CAMHB mentioned above to prepare solutions of a concentration of 256 mg/L. The resulting solutions were subjected to 2-fold dilution up to 11 times dilution to a concentration of 0.125 mg/L.

<Measurement of MIC of Flavones and Quinolones for Staphylococcus aureus>

MIC values (mg/L) of the flavones and quinolones against the strains were measured in the same manner as that of the measurement of MIC of Example 1, except that CAMHB containing each of the aforementioned flavones and quinolones prepared in Example 2 was used instead of CAMHB containing each of the test compounds. The results are shown in Table 12 below. As for the flavones, the compounds having an MIC value of 32 mg/L or lower were determined to have antibacterial activity. Further, according to the guideline of CLSI, levofloxacin having an MIC value not higher than 2 mg/L is considered to have antibacterial activity, and norfloxacin having an MIC value not higher than 8 mg/L is considered to have antibacterial activity (see, “Performance Standards for Antibacterial Susceptibility Testing; Twenty-First Informational Supplement”, Clinical and Laboratory Standards Institute, 2011, M100-S21, Vol. 31, No. 1, Replaces M100-S20 and M100-S20-U, Vol. 30, No. 1 and Vol. 30, No. 15; and “Drug Susceptibility Test and Break Points, Problems and Future View”, Ishii Yoshikazu, Japanese Journal of Chemotherapy, September 2011, Vol. 59, No. 5, pp. 454-459).

### —Calculation of MIC Ratio—

MIC ratios were calculated from the MIC values measured above in accordance with the following equation.

MIC ratio=(MIC value for S. aureus FDA209P)/(MIC value for S. aureus Mu50)

Ten kinds of the flavones mentioned in Table 11 below fall within the compounds represented by the following general formula (I), and the substituents thereof are as shown in Table 11 mentioned below.

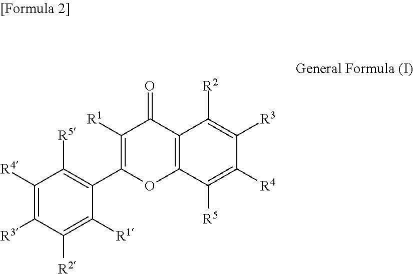

TABLE 11
Category I

| | | | | | | | | | | | | | |
|---|---|---|---|---|---|---|---|---|---|---|---|---|---|
| 7 | 4′5,7- | Apigenin | H | OH | H | OH | H | H | H | OH | H | H | Formula(5) |
| | trihydroxyflavone | | | | | | | | | | | | |
| 24 | 5-hydroxy-2-(3- | Myricetin | OCH₃ | OH | H | OCH₃ | H | H | OCH₃ | OCH₃ | OH | H | Formula(8) |
| | hydroxy-4,5- | 3,7,3′,4′- | | | | | | | | | | | |
| | dimethoxyphenyl)- | tetramethyl | | | | | | | | | | | |
| | 3,7- | ether | | | | | | | | | | | |
| | dimethoxychromen- | | | | | | | | | | | | |
| | 4-one | | | | | | | | | | | | |
| 69 | 2-(4- | Isopratol | H | H | H | OCH₃ | H | H | H | OH | H | H | Formula(7) |
| | hydroxyphenyl)-7- | | | | | | | | | | | | |
| | methoxychromen- | | | | | | | | | | | | |
| | 4-one | | | | | | | | | | | | |

Category II

| | | | | | | | | | | | | | |
|---|---|---|---|---|---|---|---|---|---|---|---|---|---|
| 6 | 3′,4′,5,7- | Luteolin | H | OH | H | OH | H | H | OH | OH | H | H | Formula(4) |
| | tetrahydroxyflavone | | | | | | | | | | | | |
| 4 | 3,4′,5,7- | Kaempferol | OH | OH | H | OH | H | H | H | OH | H | H | Formula(2) |
| | tetrahydroxyflavone | hydrate | | | | | | | | | | | |
| | hydrate | | | | | | | | | | | | |

Category III

| | | | | | | | | | | | | | |
|---|---|---|---|---|---|---|---|---|---|---|---|---|---|
| 15 | 7-methoxyflavone | 7-Methoxyflavone | H | H | H | OCH₃ | H | H | H | H | H | H | Formula(6) |
| 66 | 5-hydroxy-2-(4- | Pachypodol | OCH₃ | OH | H | OCH₃ | H | H | OCH₃ | OH | H | H | Formula(9) |
| | hydroxy-3- | | | | | | | | | | | | |
| | methoxyphenyl)- | | | | | | | | | | | | |
| | 3,7- | | | | | | | | | | | | |
| | dimethoxychromen- | | | | | | | | | | | | |
| | 4-one | | | | | | | | | | | | |
| 1 | 3,3′,4′,5,7- | Quercetin | OH | OH | H | OH | H | H | OH | OH | H | H | Formula(1) |
| | pentahydroxy- | dihydrate | | | | | | | | | | | |
| | flavone hydrate | | | | | | | | | | | | |
| 38 | 3,5,7-trihydroxy-2- | Limocitrin | OH | OH | H | OH | OCH₃ | H | OCH₃ | OH | H | H | Formula(3) |
| | (4-hydroxy-3- | | | | | | | | | | | | |
| | methoxyphenyl)-8- | | | | | | | | | | | | |
| | methoxychromen- | | | | | | | | | | | | |
| | 4-one | | | | | | | | | | | | |

Control

| | | | | | | | | | | | | | |
|---|---|---|---|---|---|---|---|---|---|---|---|---|---|
| 25 | 2-phenylchromen- | Flavone | H | H | H | H | H | H | H | H | H | H | — |
| | 4-one (2-phenyl-1- | | | | | | | | | | | | |
| | benzopyran-4- | | | | | | | | | | | | |
| | one) | | | | | | | | | | | | |

Quinolones

| | | | | | | | | | | | | | |
|---|---|---|---|---|---|---|---|---|---|---|---|---|---|
| — | levofloxacin | Levofloxacin | — | — | — | — | — | — | — | — | — | — | — |
| — | norfloxacin | Norfloxacin | — | — | — | — | — | — | — | — | — | — | — |

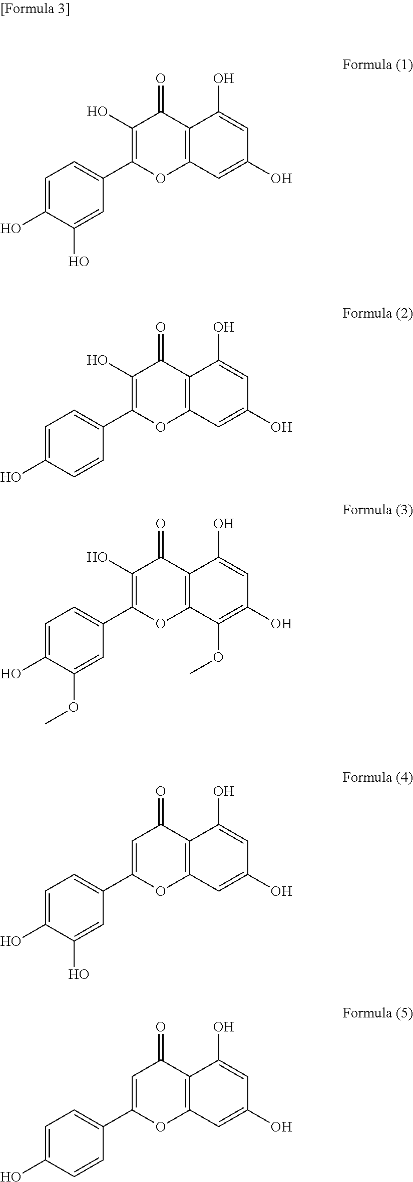 [SMILES: O=c1c(O)c(-c2ccc(O)c(O)c2)oc2cc(O)cc(O)c12 ; O=c1c(O)c(-c2ccc(O)cc2)oc2cc(O)cc(O)c12 ; COc1cc(-c2oc3c(OC)c(O)cc(O)c3c(=O)c2O)ccc1O ; O=c1cc(-c2ccc(O)c(O)c2)oc2cc(O)cc(O)c12 ; O=c1cc(-c2ccc(O)cc2)oc2cc(O)cc(O)c12]

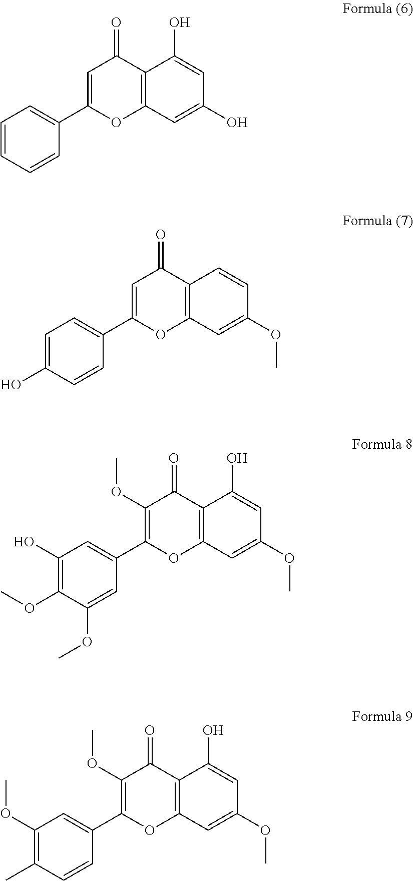

TABLE 12

| | | MIC (mg/L) | | |
|---|---|---|---|---|
| | Compound | S. aureus | | |
| No. | (Common name) | Mu50 | FDA209P | MIC ratio |

Category I

| | | | | |
|---|---|---|---|---|
| 7 | Apigenin | 4 | >128 | >32 |
| 24 | Myricetin 3,7,3′,4′- | 8 | 128 | 16 |
| | tetramethyl ether | | | |
| 69 | Isopratol | 8 | 128 | 16 |

Category II

| | | | | |
|---|---|---|---|---|
| 6 | Luteolin | 8 | 32 | 4 |
| 4 | Kaempferol hydrate | 8 | 16 | 2 |

Category III

| | | | | |
|---|---|---|---|---|
| 15 | 7-Methoxyflavone | 32 | 128 | 4 |
| 66 | Pachypodol | 32 | 128 | 4 |
| 1 | Quercetin dihydrate | 32 | 64 | 2 |
| 38 | Limocitrin | 32 | 128 | 4 |

Control

| | | | | |
|---|---|---|---|---|
| 25 | Flavone | 64 | 128 | 2 |

Quinolones

| | | | | |
|---|---|---|---|---|
| — | Levofloxacin | 8 | 0.125 | 0.016 |
| — | Norfloxacin | 64 | 0.5 | 0.008 |

From the results shown in Table 12 and on the basis of the antibacterial activities of the flavones against Staphylococcus aureus, 9 kinds of the flavones that are represented by the formulas (1) to (9) having antibacterial activities against S. aureus Mu50 in Example 1 (Nos. 1, 6, 8, 10, 26, 35, 53, 86 and 90) were classified into categories I to III as follows.

### —Category I—

The compounds having an MIC value not higher than 16 mg/L against S. aureus Mu50 with the mutated DNA gyrase A subunit and having an MIC ratio not lower than 8 were classified into Category I. It is suggested that the compounds of Category I have specific activity against the DNA gyrase A subunit. Among them, apigenin showed an extremely high antibacterial activity, i.e., an MIC ratio not lower than 32.

### —Category II—

The compounds having an MIC value not larger than 16 mg/L against S. aureus Mu50 and having an MIC ratio lower than 8 were classified into Category II. It is suggested that the target of the compounds of Category II is the DNA gyrase A subunit, however, they have low mutation specificity.

### —Category III—

The compounds, which have a low antibacterial activity against S. aureus Mu50, i.e., an MIC value of 32 mg/L, whilst have a higher antibacterial activity against S. aureus Mu50 than against S. aureus FDA209P (MIC value), were classified into Category III.

### Example 3

According to the following methods, 9 kinds of the flavones which are represented by the formulas (1) to (9) and have antibacterial activities against S. aureus Mu50 as proved in Example 1 (Nos. 1, 6, 8, 10, 26, 35, 53, 86 and 90), as well as the flavone that was revealed to have no antibacterial activity against S. aureus Mu50 (No. 37) and 2 kinds of quinolones (levofloxacin and norfloxacin) as controls were examined to know antibacterial activities against Staphylococcus aureus having the parC gene encoding topoisomerase IV and gyrA gene encoding DNA gyrase, including the mutations in the amino acid sequences of topoisomerase IV and DNA gyrase as shown in Table 13.

### <Preparation of Flavones and Quinolones>

Ten kinds of the flavones and two kinds of the quinolones shown in Table 13 below were prepared in the same manner as that of Example 2.

<Measurement of MIC of Flavones and Quinolones for Staphylococcus aureus>

MIC values of the flavones and quinolone for the strains were measured in the same manners as those of Example 1, except that S. aureus Mu50 NR-1 was also used as Staphylococcus aureus in addition to S. aureus FDA209P and S. aureus Mu50, cell suspensions of the strains were prepared. The results are shown in Table 13 below. The compounds having an MIC value not higher than 32 mg/L were determined to have antibacterial activity.

S. aureus Mu50 NR1-1 mentioned in Table 13 is a strain selected stepwise from the clinically isolated S. aureus Mu50 strain with deoxynybomycin, and it has a topoisomerase IV encoded by parC in which a phenylalanine (F) residue at the 80th position is substituted with serine (S) residue (see, Hiramatsu K. et al., J. Antimicrob. Agents, 2012, June; 39 (6), 478-85).

In Table 13, the descriptions in the parentheses after the strains indicate the mutated amino acid residue and its position, as represented by (the mutation of topoisomerase IV resulting from the mutation of pare and the mutation of DNA gyrase resulting from the mutation of gyrA). The character “w” means wild-type, representing without mutation. For example, the description (S80F, w) means that serine (S) residue at the 80th position from the amino acid terminus of the topoisomerase IV is mutated with phenylalanine (F) residue, and the DNA gyrase A subunit has no mutation.

TABLE 13

| | MIC (mg/L) | | |
|---|---|---|---|
| | Mu50 | Mu50 NR1-1 | FDA209P |
| Compound | (S80F, S84L) | (S80F, w) | (w, w) |

Category I

| | | | |
|---|---|---|---|
| Apigenin | 4 | >128 | >128 |
| Myricetin 3,7,3′,4′- | 8 | 128 | 128 |
| tetramethyl ether | | | |
| Isopratol | 16 | >128 | >128 |

Category II

| | | | |
|---|---|---|---|
| Luteolin | 8 | 32 | 32 |
| Kaempferol hydrate | 8 | 16 | 16 |

Category III

| | | | |
|---|---|---|---|
| 7-Methoxyflavone | 32 | 128 | 128 |
| Pachypodol | 32 | 128 | 128 |
| Quercetin dihydrate | 32 | 64 | 64 |
| Limocitrin | 32 | 64 | 128 |

Control

| | | | |
|---|---|---|---|
| Flavone | 64 | 128 | 128 |

Quinolones

| | | | |
|---|---|---|---|
| LVFX | 8 | 1 | 0.125 |
| NFLX | 64 | 16 | 0.5 |

From the results shown in Table 13, in the measurement of the antibacterial activity by using a set of Staphylococcus aureus strains of the same type having a mutation in gyrA, all of 9 kinds of the flavones represented by the formulas (1) to (9) (Nos. 1, 6, 8, 10, 26, 35, 53, 86, and 90) were revealed to have extremely strong antibacterial activity against S. aureus Mu50, which have become quinolone-resistant due to the mutation of gyrA in which serine (S) at the 84th position of the DNA gyrase is mutated with leucine (L) residue. These results suggest that these 9 kinds of flavones are effective as a DNA gyrase inhibitor targeting a mutant DNA gyrase.

### Example 4

In order to confirm that the flavones, which were found to have the high antibacterial activity against the Staphylococcus aureus in which the DNA gyrase was mutated (S84L) in Example 3, act on the mutant DNA gyrase as a target, a DNA cleavage assay was performed by using apigenin according to the following methods (see, Hiramatsu K., Igarashi M., Morimoto Y, Baba T., Umekita M., Akamatsu Y, Int. J. Antimicrob. Agents., 2012 June; 39(6), pp. 478-485, Epub 2012 Apr. 23; Fisher L. M., Pan X. S., Methods Mol. Med., 2008, 142, pp. 11-23; Tanaka M., Onodera Y., Uchida Y., Sato K., Hayakawa I., Antimicrob. Agents Chemother., 1997; 41, pp. 2362-2366).

### <Preparation of Enzyme Solution>

### —Preparation of Wild-Type GyrA Expression Vector—

The gene sequence encoding the wild-type GyrA of Staphylococcus aureus (henceforth also referred to as “GyrA(wt)”) shown as SEQ ID NO: 1 was cloned into the plasmid pMAL-c2 (produced by New England Biolabs) (the resultant is henceforth also referred to as “pMAL-c2-gyrA”).

### —Preparation of Wild-Type GyrB Expression Vector—

The gene sequence encoding the wild-type GyrB of Staphylococcus aureus (henceforth also referred to as “GyrB(wt)”) shown as SEQ ID NO: 2 was cloned into the plasmid pMAL-c2 (produced by New England Biolabs) (the resultant is henceforth also referred to as “pMAL-c2-gyrB”).

### Preparation of (a) GyrA(Wt), (b) GyrB(Wt), and (c) Mutant GyrA(S84L)—

E. coli BL21(DE3) (produced by Invitrogen) was transformed with pMAL-c2-gyrA mentioned above, with pMAL-c2-gyrB mentioned above, or with pMAL-c2-SAGAmut84 containing a mutant gyrA encoding the amino acid sequence of GyrA(wt) in which serine residue at the 84th position from the amino terminus is substituted with a leucine residue (see, Hiramatsu K., Igarashi M., Morimoto Y, Baba T., Umekita M., Akamatsu Y., Int. J. Antimicrob. Agents, 2012 June; 39(6), 478-85). To the culture medium of the resulting transformed Escherichia coli, 1 mM isopropyl-6-thiogalactopyranoside (produced by Wako Pure Chemical Industries) was added to induce expression of maltose-binding GyrA(wt), maltose-binding GyrB(wt), or maltose-binding mutant GyrA(S84L), and the bacterium was cultured overnight at 25° C. After completion of the culture, the cells were collected, and suspended in a buffer (20 mM Tris-HCl (pH 8.0), 200 mM sodium chloride, 1 mM ethylenediaminetetraacetic acid (EDTA), 1 mM dithiothreitol (DTT)), a protein extracting reagent (10× BugBuster (registered trademark), produced by Novagen) in a volume of 1/10 of the volume of the buffer, and 2.5 μL/10 mL of the buffer of Benzonase (registered trademark) nuclease (produced by Novagen) were added, the mixture was left standing at 25° C. for 30 minutes, and the cells were disrupted and lysed to obtain a disrupted cell suspension containing the maltose-binding GyrA(wt), maltose-binding GyrB(wt), or maltose-binding mutant GyrA(S84L).

Then, each disrupted cell suspension mentioned above was added to a column filled with an amylose resin covalently bound with maltose (produced by Novagen). A buffer (20 mM Tris-HCl (pH 8.0), 200 mM sodium chloride, 1 mM EDTA) containing 10 mM maltose was added to the column to elute the maltose-binding GyrA(wt), maltose-binding GyrB(wt), and maltose-binding mutant GyrA(S84L).

To each eluate, 4 units/1 mL of the eluate of protease (Factor Xa, produced by Amersham) was added, and the mixture was left standing at 25° C. for 3 hours and 30 minutes to cleave the maltose-binding proteins, GyrA(wt), GyrB(wt), and mutant GyrA(S84L) from the maltose-binding GyrA(wt), maltose-binding GyrB(wt), and maltose-binding mutant GyrA(S84L).

Then, GyrA(wt), GyrB(wt), and mutant GyrA(S84L) were concentrated by using an ultrafiltration unit (YM-50 Filter Unit, produced by Millipore).

The concentrated GyrA(wt), GyrB(wt), and mutant GyrA(S84L) were each suspended in a buffer (35 mM Tris-HCl (pH 7.5), 24 mM potassium chloride, 6 mM magnesium chloride, 1.8 mM spermidine, 0.36 mg/mL bovine serum albumin, 5 mM DTT, 50 volume % glycerol) at a concentration of 1 μg/μL for GyrA(wt), 2 μg/μL for GyrB(wt), or 1 μg/μL for mutant GyrA(S84L).

### <DNA Cleavage Assay>

—Cleavage of Plasmid DNA with GyrA(Wt) and GyrB(Wt)—

A substrate plasmid DNA, pTWV228 (0.4 μg, produced by Takara Bio), and levofloxacin (LVFX) or apigenin were added to a buffer (35 mM Tris-HCl (pH 7.5), 24 mM potassium chloride, 6 mM magnesium chloride, 1.8 mM spermidine, 0.36 mg/mL bovine serum albumin, 5 mM DTT), and 0.15 μL of the enzyme solution of GyrA(wt) and 0.2 μL of the enzyme solution of GyrB(wt) were added to the mixture to prepare a reaction mixture in a total volume of 20 μL.

Levofloxacin was added at a final concentration of 0, 0.5, 1, 2, 4, 8, 16, or 32 μg/mL. Apigenin was added at a final concentration of 0, 2, 4, 8, 16, 32, 64, or 128 μg/mL.

The aforementioned reaction solution was left standing at 25° C. for 30 minutes to allow the reaction. Subsequently, a 6 mass % solution of sodium laurylsulfate (2 μL) and a 5 mg/mL solution of Proteinase K (2 μL, produced by Wako Pure Chemical Industries) were added to the reaction solution, and the mixture was left standing at 37° C. for 30 minutes to separate GyrA(wt) and GyrB(wt) bound to DNA.

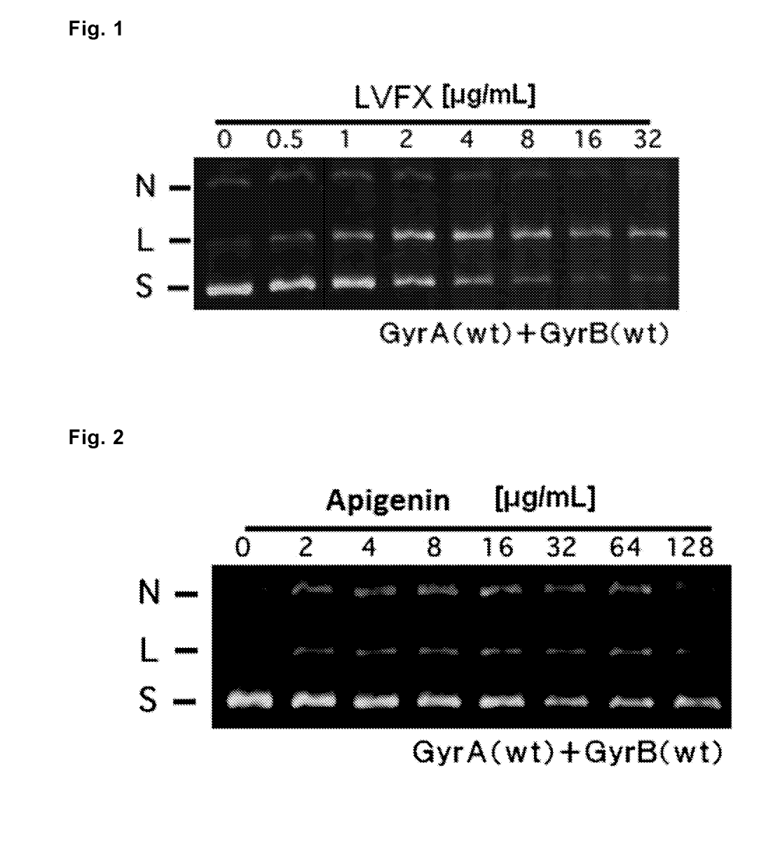

Then, the whole volume of the solution in which GyrA(wt) and GyrB(wt) were cleaved was subjected to electrophoresis on 1 mass % agarose gel to visualize the resultants of the reaction. The results are shown in FIGS. 1 and 2. In FIGS. 1 and 2, “S” represents the substrate plasmid DNA having a superhelix structure, “L” represents linear double-stranded DNA formed by cleavage of the aforementioned substrate plasmid DNA with the DNA gyrase, and “N” represents double-stranded DNA in which one strand of the double-strand substrate plasmid DNA was cleaved by the DNA gyrase.

Further, intensities of the bands in the visualized images shown in FIGS. 1 and 2 were quantified by using software (ImageJ v. 10.2, National Institutes of Health, http://rsb.info.nih.gov/ij/), and IC₅₀ values were calculated by the method of Fisher L. M., Pan X. S., Antimicrob. Agents Chemother., 1999, 43(5), 1129-36.

Specifically, the intensities of the bands of “S”, “L”, and “N” were measured for each concentration of levofloxacin or apigenin by using the aforementioned software. Then, the total value of the intensities of the bands of “S”, “L”, and “N” was calculated for each concentration, and the result was used as “total DNA amount” (equation 1 mentioned below). Further, the total value of the intensities of the bands of “L” and “N” was also calculated, and the result was used as “amount of cleaved DNA” (equation 2 mentioned below). Then, the ratio (%) of the amount of the substrate plasmid DNA to the total DNA amount mentioned above was calculated in accordance with the following equation 3, and the ratio (%) of the amount of the cleaved DNA to the total DNA amount mentioned above was calculated in accordance with the following equation 4. IC₅₀ value of levofloxacin or apigenin was calculated from the ratio of the cleaved DNA. The results are shown in Table 14 below.

Total DNA amount=S+L+N (Equation 1)

Amount of cleaved DNA=L+N (Equation 2)

Ratio of substrate plasmid DNA (%)=S/(S+L+N)×100 (Equation 3)

Ratio of cleaved DNA amount (%)=(L+N)/(S+L+×100 (Equation 4)

### <DNA Cleavage Assay>

—Cleavage of Plasmid DNA with Mutants GyrA(S84L) and GyrB(Wt)—

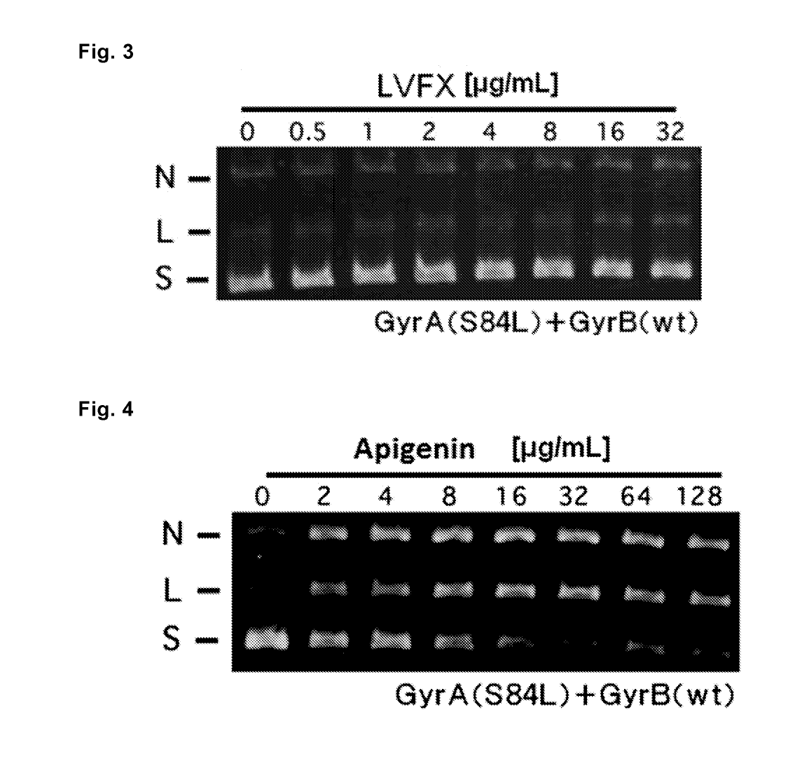

An assay was performed in the same manner as that of the cleavage of the plasmid DNA with GyrA(wt) and GyrB(wt) mentioned above, provided that 0.15 μL, of an enzyme solution of mutant GyrA(S84L) and 0.2 μL of the enzyme solution of GyrB(wt) were added instead of 0.154 of the enzyme solution of GyrA(wt) and 0.2 μL of the enzyme solution of GyrB(wt). The results of the agarose gel electrophoresis are shown in FIGS. 3 and 4. In FIGS. 3 and 4, the symbols “S”, “L”, and “N” have the same meanings as those explained for FIGS. 1 and 2.

IC₅₀ value of levofloxacin or apigenin was also calculated from the visualized images shown in FIGS. 3 and 4 in the same manner as that used for the calculation using the results shown in FIGS. 1 and 2. The results are shown in Table 15 shown below.

TABLE 14

| | | GyrA(wt) + GyrB(wt) | | |
|---|---|---|---|---|
| | | Ratio of | | |
| | | substrate | Ratio of | |
| | Concentration | plasmid DNA | cleaved DNA | IC₅₀ |
| Compound | [μg/mL] | (%) | (%) | (μg/mL) |
| LVFX | 0 | 95 | 5 | 2 |
| | 0.5 | 68 | 32 | |
| | 1 | 57 | 43 | |
| | 2 | 42 | 58 | |
| | 4 | 39 | 61 | |
| | 8 | 36 | 64 | |
| | 16 | 30 | 70 | |
| | 32 | 33 | 67 | |
| Apigenin | 0 | 94 | 6 | >128 |
| | 2 | 66 | 34 | |
| | 4 | 65 | 35 | |
| | 8 | 64 | 36 | |
| | 16 | 65 | 35 | |
| | 32 | 60 | 40 | |
| | 64 | 57 | 43 | |
| | 128 | 61 | 39 | |

S: Substrate plasmid DNA having superhelix structure
L: Linear double-stranded DNA formed by cleavage of the substrate plasmid DNA
N: Double-stranded DNA formed by cleavage of one strand of the double-stranded substrate plasmid DNA

TABLE 15

| | | GyrA(S84L) + GyrB(wt) | | |
|---|---|---|---|---|
| | | Ratio of | | |
| | | substrate | Ratio of | |
| | Concentration | plasmid DNA | cleaved DNA | IC₅₀ |
| Compound | [μg/mL] | (%) | (%) | (μg/mL) |
| LVFX | 0 | 94 | 6 | >32 |
| | 0.5 | 85 | 15 | |
| | 1 | 85 | 15 | |
| | 2 | 82 | 18 | |
| | 4 | 82 | 18 | |
| | 8 | 81 | 19 | |
| | 16 | 87 | 13 | |
| | 32 | 78 | 22 | |
| Apigenin | 0 | 96 | 4 | 8 |
| | 2 | 53 | 47 | |
| | 4 | 54 | 46 | |
| | 8 | 46 | 54 | |
| | 16 | 37 | 63 | |
| | 32 | 11 | 89 | |
| | 64 | 35 | 65 | |
| | 128 | 18 | 82 | |

S: Substrate plasmid DNA having superhelix structure
L: Linear double-stranded DNA formed by cleavage of the substrate plasmid DNA
N: Double-stranded DNA formed by cleavage of one strand of the double-stranded substrate plasmid DNA

The DNA cleavage assay is a method for evaluating an inhibitory action of a flavone or quinolone for DNA gyrase by detecting whether or not DNAs cleaved by DNA gyrase are recombined. Specifically, in FIGS. 1 to 4, when, a DNA gyrase inhibitory activity of levofloxacin or apigenin is observed, “S” decreases in a levofloxacin or apigenin concentration-dependent manner, the ratios of “N” and “L” correspondingly increase, and in Tables 14 and 15, the ratio of the cleaved DNA increases.

As seen from the results shown in FIGS. 1 and 3, levofloxacin exhibited a concentration-dependent inhibitory activity against the wild-type DNA gyrase (FIG. 1), whilst it exhibited no inhibitory activity against the mutant DNA gyrase (FIG. 3).

In contrast, apigenin did not exhibit any inhibitory activity against the wild-type DNA gyrase (FIG. 2), whilst it exhibited a concentration-dependent inhibitory activity against the mutant DNA gyrase (FIG. 4).

Further, from the results shown in Tables 14 and 15, the IC₅₀ values of apigenin indicating binding affinity for the DNA gyrase and the substrate plasmid DNA at the time of formation of the complex of these were higher than 128 μg/mL (higher than 473.7 μM) when GyrA(wt) and GyrB(wt) were used as the enzyme solutions, and about 8 μg/mL (29.6 μM) when the mutant GyrA(S84L) and GyrB(wt) were used. Therefore, apigenin is suggested to have a binding inhibitory activity specific to GyrA(S84L).

### Example 5

Emergence frequency of levofloxacin-resistant Staphylococcus aureus was examined in the presence or absence of apigenin by using a levofloxacin-susceptible Staphylococcus aureus strain according to the following method.

### <Preculture of Bacterium>

The levofloxacin-susceptible Staphylococcus aureus strain MS5935-1 (strain in which the 80th serine residue from the amino terminus of the wild-type topoisomerase IV encoded by parC is mutated with phenylalanine residue, see, Hiramatsu K., Igarashi M., Morimoto Y, Baba T., Umekita M., Akamatsu Y., Int. J. Antimicrob. Agents, 2012 June; 39(6), 478-85) was cultured overnight at 37° C. with shaking in 4 mL of TBS (produced by Dickinson). After completion of the culture, the cells were suspended in fresh TBS, and the cell suspension was adjusted to have an absorbance of 0.3 measured at 578 nm. Then, the cell suspension was diluted 10,000 times with TBS. This diluted cell suspension was inoculated (0.1 mL each) into ten test tubes containing 4 mL each of TBS, and preculture was carried out overnight at 37° C.

The MIC value of levofloxacin for S. aureus MS5935-1 obtained by the same method as that used for the measurement of MIC for Staphylococcus aureus mentioned in Example 2 was 0.5 mg/L, and the MIC value of apigenin similarly obtained was higher than 128 mg/L.

### <Preparation of Agar Plate Medium>

### —Preparation of Agar Plate Medium Containing Levofloxacin—

Levofloxacin was dissolved in sterilized water at a concentration of 40 mg/L. This solution of levofloxacin was added on plates in a volume of 1 mL/plate, and 9 mL of M-H (Mueller Hinton) Agar (produced by Becton, Dickinson and Company) was added to each plate. Five of such plates were prepared.

The final concentration of levofloxacin in the levofloxacin-containing agar plate medium was 4 mg/L.

### Preparation of Agar Plate Medium Containing Apigenin and Levofloxacin—

Apigenin was dissolved in DMSO at a concentration of 640 mg/L. Further, levofloxacin was dissolved in sterilized water at a concentration of 80 mg/L. Each of the solutions of apigenin and levofloxacin was added on plates in a volume of 0.5 mL/plate, and 9 mL of M-H (Mueller Hinton) Agar (produced by Becton, Dickinson and Company) was added to each plate. Five of such plates were prepared.

The final concentrations of apigenin and levofloxacin in the apigenin and levofloxacin-containing agar plate medium were 32 mg/L and 4 mg/L, respectively.

### <Culture of Bacterium on Agar Medium>

The aforementioned preculture medium was concentrated by centrifugation to a volume of 150 μL, 15 μL of the concentrate was separated for colony counting, the whole volume of the remaining medium was inoculated onto the aforementioned agar plate medium, and culture was performed at 37° C. for 2 days.

<Calculation of Emergence Frequency of Levofloxacin-Resistant Staphylococcus aureus>

Number of the colonies formed on each agar plate medium after the culture of 2 days was counted. The emergence frequency of levofloxacin-resistant Staphylococcus aureus was calculated according to the following equation using the number of colonies and the number of bacterial cells inoculated onto each agar plate medium (CFU, colony forming unit). Further, from the calculated emergence frequency of the levofloxacin-resistant Staphylococcus aureus, emergence frequency of levofloxacin resistance mutation and 95% confidence interval (CI range) were calculated by using FALCOR (Fluctuation AnaLysis CalculatOR, http://www.keshaysingh.org/protocols/FALCOR.html, see, Hall B. M. et al., Bioinformatics., 2009, 25(12), 1564-5). The results are shown in Table 16 below. Emergence frequency of levofloxacin-resistant Staphylococcus aureus=(Number of grown colonies)/(Number of cells inoculated onto levofloxacin-containing agar plate medium or apigenin and levofloxacin-containing agar plate medium)

TABLE 16

| Agar | Compound | | Number of | | Emergence frequency | Emergence frequency of | |
|---|---|---|---|---|---|---|---|
| plate | concentration in agar | | inoculated | Number of | of levofloxacin- | levofloxacin resistance | |
| medium | plate medium (mg/L) | | cells (×10⁹ | grown colonies | resistant bacteria | mutation | 95% CI range (upper |
| No. | LVFX | Apigenin | CFU) | (colonies) | (×10⁻⁹) | (mutations/genera) | bound/lower bound) |
| 1 | 4 | 0 | 12.3 | 83 | 6.74 | 1.45 × 10⁻⁹ | 2.11/0.89 |
| 2 | 4 | 0 | 14.7 | 78 | 5.31 | | |
| 3 | 4 | 0 | 16.7 | 66 | 3.96 | | |
| 4 | 4 | 0 | 8.6 | 33 | 3.86 | | |
| 5 | 4 | 0 | 12.0 | 328 | 27.3 | | |
| 1 | 4 | 32 | 14.9 | 4 | 0.27 | 0.05 × 10⁻⁹ | 0.1158/0.0086 |
| 2 | 4 | 32 | 14.6 | 0 | <0.07 | | |
| 3 | 4 | 32 | 12.5 | 0 | <0.08 | | |
| 4 | 4 | 32 | 12.6 | 0 | <0.08 | | |
| 5 | 4 | 32 | 12.5 | 14 | 1.12 | | |

From the results shown in Table 16, when the bacterium was cultured on the agar plate medium containing only levofloxacin (levofloxacin-containing agar plate medium), the emergence frequency of levofloxacin resistance mutation was 1/29 of that observed when the bacterium was cultured on the agar plate medium containing levofloxacin and apigenin (Emergence frequency of levofloxacin resistance mutation on the agar plate medium containing levofloxacin and apigenin/Emergence frequency of levofloxacin resistance mutation on the agar plate medium containing levofloxacin=1/29), and thus significantly lower than the latter.

This result suggests that the flavones classified into the category I in Example 2 including apigenin can suppress emergence of quinolone-resistant Staphylococcus aureus, when they are used together with a quinolone.

The results of Examples 1 to 5 suggest that the flavones represented by the structural formulas (1) to (9) have inhibitory activity against the DNA gyrase of Staphylococcus aureus, and further have antibacterial activity complementary to that of quinolones, and therefore they are useful as an reverse antibacterial agent.

The S84L mutation of the DNA gyrase is the most frequently occurring mutation found in clinically separated quinolone-resistant MRSA and Staphylococcus aureus strains (see, Hiramatsu K., Igarashi M., Morimoto Y., Baba T., Umekita M., Akamatsu Y, Int. J. Antimicrob. Agents, 2012 June; 39(6), 478-85). Therefore, it was found that the flavones represented by the formulas (1) to (9) that exhibit effectiveness against Staphylococcus aureus strains having the S84L mutation in the DNA gyrase can specifically inhibit the growth of quinolone-resistant Staphylococcus aureus strains, and can further suppress emergence of quinolone-resistant Staphylococcus aureus due to use of quinolones.

### Example 6

J-Q20 was synthesized according to the following scheme.

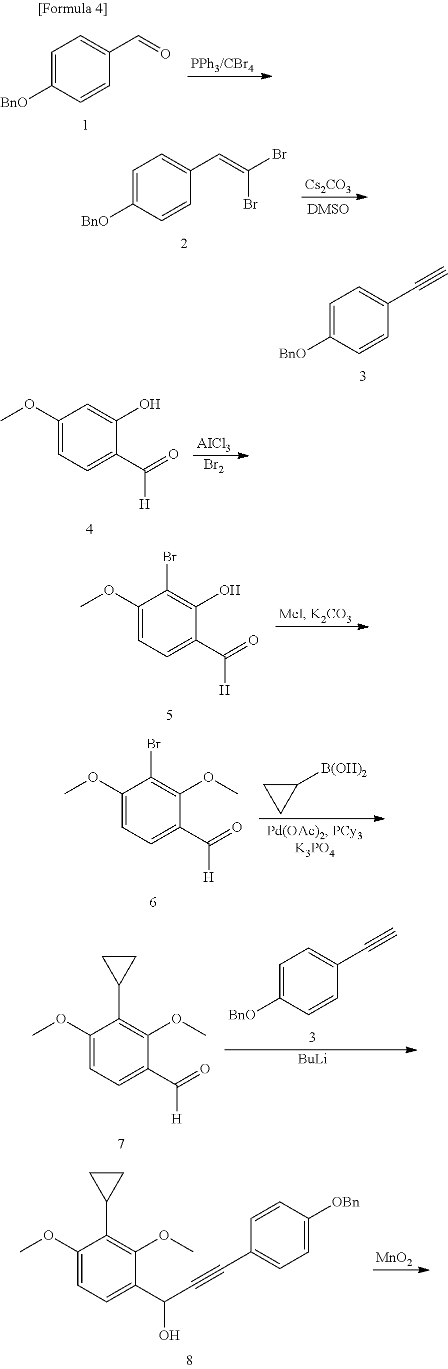

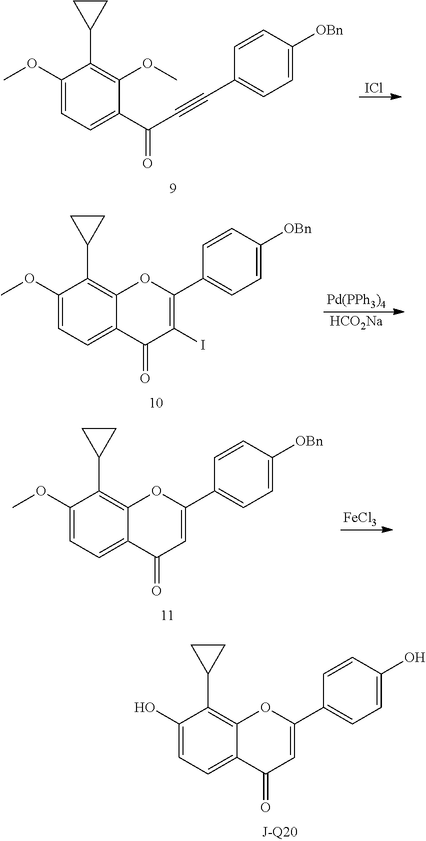

### (a) 4-(2,2-Dibromovinyl)-1-(phenylmethoxy)benzene (2)

To a solution of CBr₄ (15.7 g, 47.2 mmol) in dichloromethane (200 mL) was added triphenylphosphine (24.7 g, 94.4 mmol) at 0° C. under nitrogen atmosphere. After completion, a solution of the compound 1 (5.0 g, 23.6 mmol) in dichloromethane (20 mL) was added dropwise to the mixture at 0° C. under nitrogen atmosphere. The reaction mixture was warmed to room temperature and stirred for 30 minutes. Water (300 mL) was poured into the reaction mixture and extracted with dichloromethane (2×200 mL) The combined organic layers were washed with brine (500 mL), dried over Na₂SO₄, and concentrated in vacuo. The residue was purified by silica gel column chromatography (petroleum ether/ethyl acetate=10:1) to afford the compound 2 (9.22 g, yield 96.5%) as a white solid.

¹H NMR (CDCl₃, 300 MHz): δ 5.08 (s, 2H), 6.94-6.97 (m, 2H), 7.33-7.44 (m, 5H), 7.49-7.52 (m, 2H).

### (b) 4-Ethynyl-1-(phenylmethoxy)benzene (3)

To a solution of the compound 2 (8.29 g, 22.7 mmol) in dimethylsulfoxide (DMSO, 100 mL) was added Cs₂CO₃ (22.2 g, 68.0 mmol) and heated to 110° C. overnight. Then the reaction mixture was cooled to room temperature and poured into water (500 mL), and extracted with ethyl acetate (2×300 mL). The combined organic layers were washed with brine (500 mL), dried over Na₂SO₄, and concentrated in vacuo. The residue was purified by silica gel column chromatography (petroleum ether/ethyl acetate=50:1) to afford the compound 3 (3.17 g, yield: 68%) as a white solid.

¹H NMR (CDCl₃, 300 MHz): δ 3.00 (s, 1H), 5.07 (s, 2H), 6.90-6.94 (m, 2H), 7.33-7.455 (m, 7H).

### (c) 3-Bromo-2-hydroxy-4-methoxybenzaldehyde (5)

A solution of the compound 4 (3.04 g, 20.0 mmol) in dichloromethane (120 mL) was cooled to −20° C. Then aluminum chloride (2.66 g, 20.0 mmol) was added in three portions. The suspension was stirred for 15 minutes before a solution of bromine (3.2 g, 20.0 mmol) in dichloromethane (20 mL) was added over 20 minutes. It was stirred overnight while warming to room temperature. Then a saturated solution of Na₂SO₃ (30 mL) and hydrochloric acid (4 M, 60 mL) was added in order. The aqueous layer was extracted two times with dichloromethane (30 mL each). It was dried over magnesium sulfate, filtered, and concentrated in vacuo. After purification by column chromatography, the compound 5 was isolated as a white solid (2.73 g, 61%).

¹H NMR (CDCl₃, 300 MHz): δ 3.99 (s, 3H), 6.60-6.63 (d, J=8.7 Hz, 1H), 7.49 (d, J=8.7 Hz, 1H), 9.71 (s, 1H), 11.9 (s, 1H).

### (d) 3-Bromo-2,4-dimethoxybenzaldehyde (6)

A mixture of the compound 5 (1.00 g, 4.35 mmol), iodomethane (1.23 g, 8.69 mmol) and K₂CO₃ (1.2 g, 8.69 mmol) in dry dimethylformamide (15 mL) was stirred at room temperature overnight. The reaction mixture was poured into water (50 mL) and extracted with ethyl acetate (3×30 mL). The combined organic layers were washed with brine (50 mL), dried over Na₂SO₄, and concentrated in vacuo to afford the compound 6 (890 mg, 84%) as a white solid.

¹H NMR (CDCl₃, 300 MHz): δ 3.98 (s, 6H), 6.79 (d, J=8.7 Hz, 1H), 7.86 (d, J=8.7 Hz, 1H), 10.22 (s, 1H).

### (e) 3-Cyclopropyl-2,4-dimethoxybenzaldehyde (7)

To a mixture of a solution of the compound 6 (2.45 g, 10.0 mmol), cyclopropylboronic acid (1.29 g, 15.0 mmol), tricyclohexylphosphonium tetrafluoroborate (368 mg, 1.00 mmol), K₃PO₄ (7.42 g, 35.0 mmol) in toluene (35 mL) and water (2 mL) was added Pd(OAc)₂ (154 mg, 0.5 mmol) and stirred at 95° C. under nitrogen atmosphere overnight. The mixture was cooled to room temperature and filtered. The filtrate was poured into water (100 mL), and extracted with ethyl acetate (2×50 mL). The combined organic layers were washed with brine (100 mL), dried over Na₂SO₄ and concentrated in vacuo. The residue was purified by column chromatography to afford the compound 7 (1.6 g, yield 78%) as a colorless oil.

¹H NMR (CDCl₃, 300 MHz): δ 0.85-0.94 (m, 4H), 1.70-1.74 (m, 1H), 3.86 (s, 3H), 3.93 (s, 3H), 6.67 (d, J=8.7 Hz, 1H), 7.69 (d, J=8.7 Hz, 1H), 10.22 (s, 1H).

### (f) 1-(3-Cyclopropyl-2,4-dimethoxyphenyl)-3-[4-(phenylmethoxy)phenyl]prop-2-yn-1-ol (8)

To a solution of the compound 3 (1.21 g, 5.83 mmol) in tetrahydrofuran (THF, 20 mL) was added n-BuLi (2.4 mL, 2.5 M in THF) dropwise at −65° C. under nitrogen atmosphere. After stirred for 0.5 hours at this temperature, a solution of the compound 7 (1.00 g, 4.85 mmol) in THF (2 mL) was added slowly to maintain the temperature between −65° C. and −60° C. Then the mixture was warmed to ambient temperature, and the reaction was quenched with saturated NH4C₁ aqueous. The aqueous layer was extracted with ethyl acetate (50 mL×2). The combined organic layers were washed with brine, dried over Na₂SO₄, and concentrated to give a crude product of the compound 8 (2.28 g) as a yellow solid, which was used directly in the next step.

### (g) 1-(3-Cyclopropyl-2,4-dimethoxyphenyl)-3-[4-(phenylmethoxy)phenyl]prop-2-yn-1-one (9)

A mixture of the compound 8 (2.28 g, 5.50 mmol) and MnO₂ (2.40 g, 27.5 mmol) in chloroform (25 mL) was refluxed overnight under nitrogen atmosphere. After the reaction mixture was filtered, the filtrate was concentrated, and the residue was purified by column chromatography (petroleum ether/ethyl acetate=10:1) to give the compound 9 (1.5 g, 66%) as a yellow oil.

¹H NMR (300 MHz, CDCl₃): δ 0.92-0.94 (m, 4H), 1.77-1.82 (m, 1H), 3.88 (s, 3H), 3.95 (s, 3H), 5.10 (s, 2H), 6.67 (d, J=9.0 Hz, 1H), 6.96-7.00 (m, 2H), 7.33-7.43 (m, 5H), 7.57-7.61 (m, 2H), 7.96 (d, J=9.0 Hz, 1H).

### (h) 8-Cyclopropyl-3-iodo-7-methoxy-2-[4-(phenylmethoxy)phenyl]chromen-4-one (10)

The compound 9 (1.5 g, 3.64 mmol) and ICl (5.46 mL, 5.46 mmol) were each dissolved in dry dichloromethane (25 mL) and cooled to −70° C. to give solutions A and B respectively. The solution B was added to the solution A over 20 minutes and stirred for 1.5 hours at this temperature. Saturated Na₂S₂O₃ aqueous (30 mL) and NaCl (40 mL) were added. The organic layer was dried over Na₂SO₄, and concentrated, and the residue was purified by silica gel column chromatography (petroleum ether:ethyl acetate=10:1) to give the compound 10 (1.8 g, 95%) as a yellowish solid.

¹H NMR (300 MHz, CDCl₃): δ 0.92-0.96 (m, 4H), 1.88-1.92 (m, 1H), 3.94 (s, 3H), 5.15 (s, 2H), 6.97 (d, J=9.3 Hz, 1H), 7.09-7.13 (m, 2H), 7.35-7.48 (m, 5H), 7.84-7.87 (m, 2H), 8.10 (d, J=9.0 Hz, 1H).

### (i) 8-Cyclopropyl-7-methoxy-2-[4-(phenylmethoxy)phenyl]chromen-4-one (11)

A solution of the compound 10 (524 mg, 1.00 mmol), sodium formate (208 mg, 2.00 mmol) and Pd(PPh₃)₂Cl₂ (35 mg, 0.05 mmol) in DMF (8 mL) was stirred at 95° C. under nitrogen atmosphere for 2 hours. TLC showed the reaction was completed. Ethyl acetate (100 mL) and water (100 mL) were added. The organic phase was washed with water (100 mL) and brine, dried over Na₂SO₄, and concentrated in vacuo. The residue was slurried with 1-methoxy-1,1-dimethylethane (MTBE) to afford the compound 11 (400 mg, 100%) as a yellow solid.

¹H NMR, (300 MHz, CDCl₃): δ 0.88-1.09 (m, 4H), 1.93-2.02 (m, 1H), 3.90 (s, 3H), 5.21 (s, 2H), 6.83 (s, 1H), 7.14-7.22 (m, 3H), 7.30-7.47 (m, 5H), 7.86 (d, J=8.7 Hz, 1H), 8.02-8.06 (m, 2H).

### (j) 8-Cyclopropyl-7-hydroxy-2-(4-hydroxyphenyl)chromen-4-one (J-Q21)

A solution of NaH (259 mg, 60%, 13.3 mmol) and ethanethiol (1.0 mL, 13.6 mmol) in dry DMF (20 mL) was stirred for 30 minutes at room temperature, a solution of the compound 11 (750 mg, 1.89 mmol) in DMF (15 mL) was added, and heated at 150° C. overnight. The reaction mixture was cooled to room temperature, poured into water (100 mL), and extracted with ethyl acetate (2×80 mL). The combined organic layers were washed with brine (100 mL), dried over Na₂SO₄ and concentrated in vacuo. The residue was purified by silica gel column chromatography (petroleum ether/ethyl acetate) to give J-Q21 (120 mg, 21%) as a white solid.

¹H NMR: (300 MHz, DMSO-d₆): δ 0.90-1.06 (m, 4H), 1.91-1.98 (m, 1H), 6.70 (s, 1H), 6.88-6.94 (m, 3H), 7.68 (d, J=8.7 Hz, 1H), 7.90-7.95 (m, 2H), 10.22 (br s, 1H), 10.37 (br s, 1H).

### Example 7

J-Q12.1 was synthesized according to the following scheme.

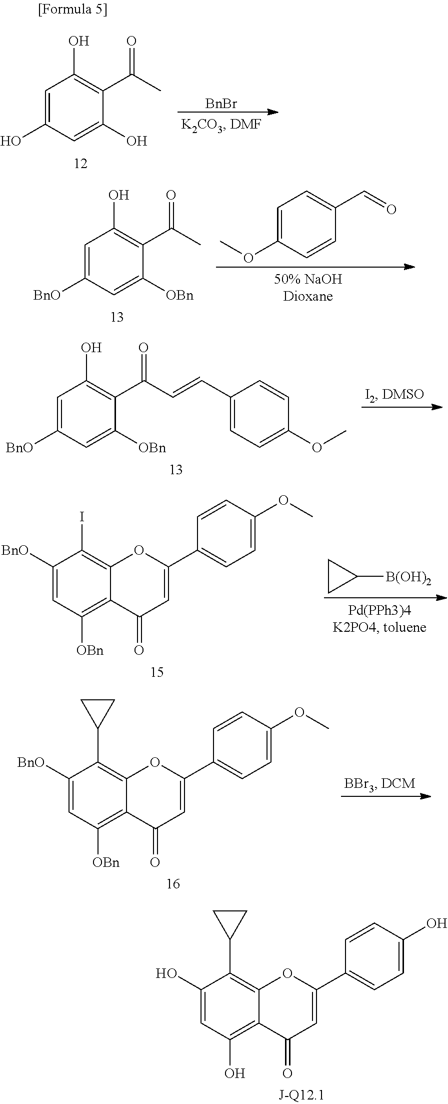

### (a) 1-(2,4-Bis-benzyloxy-6-hydroxyphenyl)-ethanone (13)

To a solution of the compound 12 (8.00 g, 43.0 mmol) and K₂CO₃ (13.7 g, 99.1 mmol) in DMF (100 mL) was added benzyl bromide (15.1 g, 88.3 mmol), and the mixture was stirred at room temperature overnight. DMF was removed under vacuum. Ethyl acetate and water was added. The organic layer was washed with brine, dried over Na₂SO₄, and concentrated to give a residue. The residue was purified by silica gel chromatography (ethyl acetate:petroleum ether=40:1) to give a crude product of the compound 13 (7.55 g) as a yellow solid.

### (b) 1-(2,4-Bisbenzyloxy-6-hydroxyphenyl)-3-(4-methoxyphenyl)propen-one (14)

To a solution of the compound 13 (4.00 g, 11.5 mmol) in dioxane (40 mL) was added 50% NaOH (40 mL), and the mixture was stirred at 30° C. for 30 minutes. A solution of 4-methoxybenzaldehyde in dioxane was added, and stirred at 30° C. for 16 hours. 3 N HCl was added to adjust pH=6 to 7, and ethyl acetate was added to extracted the product (100 mL×3). The combined organic layers were washed with water (100 mL) and brine (100 mL), dried over anhydrous NaSO₄, and concentrated in vacuo, and the residue was purified by silica gel chromatography (ethyl acetate:petroleum ether=10:1) to afford a crude product of the compound 14 (4.54 g) as a yellow solid.

### (c) 5,7-Bisbenzyloxy-8-iodo-2-(4-methoxyphenyl)-chromen-4-one (15)

To a solution of the compound 14 (4.54 g, 8.37 mmol) in anhydrous DMSO (300 mL) was added 12 (4.88 g, 19.2 mmol) under a nitrogen atmosphere. After allowed to stir at 145° C. for 15 hours, the mixture was cooled to room temperature. Ethyl acetate (1000 mL) was added, and the reaction was quenched with saturated Na₂SO₃ aqueous. Then the organic layer was washed with water and brine (100 mL×4). The organic layer was dried over Na₂SO₄, and concentrated in vacuo, and the residue was purified by silica gel column chromatography (dichloromethane:petroleum ether=1:2) to give the compound 15 as a yellow oil (900 mg).

¹H NMR (300 MHz, CDCl₃): δ 5.15 (2H, s), 5.26 (2H, s), 6.57 (1H, s), 6.53 (1H, s), 7.08 (2H, d, J=8.7 Hz), 7.36-7.45 (8H, m), 7.52 (2H, d, J=7.5 Hz), 7.82 (2H, d, J=8.4 Hz).

### (d) 5,7-Bisbenzyloxy-8-cyclopropyl-2-(4-methoxyphenyl)-chromen-4-one (16)

A solution of the compound 15 (1.00 g, 1.73 mmol), cyclopropylboronic acid (2.00 g, 23.3 mmol), Pd(PPh₃)₄ (200 mg), and KsPO₄.3H₂O (2.0 g, 7.6 mmol) in toluene (300 mL) was heated up to 80° C. and stirred for 24 hours under nitrogen atmosphere. After cooling down to room temperature, the mixture was diluted with ethyl acetate (300 mL) and adjusted to pH=6 to 7 with 2 N HCl. The organic layer was washed with water (100 mL) and brine (100 mL), dried over anhydrous Na₂SO₄ and concentrated in vacuo. The residue was purified by silica gel column chromatography (ethyl acetate:petroleum ether=5:1) to afford the compound 16 (500 mg, yield: 18%) as a yellow solid.

¹H NMR (300 MHz, CDCl₃): δ 0.88-0.92 (2H, m), 1.01-1.06 (2H, m), 1.83-1.89 (1H, m), 5.15 (2H, s), 5.17 (2H, s), 6.53 (1H, s), 6.56 (1H, s), 7.06 (2H, d, J=9.0 Hz), 7.34-7.52 (10H, m), 7.82 (2H, d, J=9.3 Hz), 13.10 (1H, s).

### (e) 8-Cyclopropyl-5,7-dihydroxy-2-(4-hydroxyphenyl)-chromen-4-one (J-Q12.1)

To a solution of the compound 16 (500 mg, 1.02 mmol) in dichloromethane (50 mL) cooled to −70° C. was added 1 N solution of BBr₃ in dichloromethane (15 mL). The mixture was stirred at room temperature for 2 hours. Methanol was added to quench the reaction. The mixture was then adjusted to pH=5 to 6 with saturated sodium hydrogencarbonate aqueous, and the aqueous layer was extracted with dichloromethane (50 mL×3). The combined organic layers were washed with brine (50 mL), dried over anhydrous Na₂SO₄, and concentrated in vacuo. The residue was purified by Prep-HPLC and lyophilized to afford the compound J-Q12.1 (93 mg, yield: 46%) as a yellow solid.

¹H NMR (300 MHz, CDCl₃): δ 0.80-0.85 (2H, m), 0.96-1.00 (2H, m), 3.29-3.34 (1H, m), 6.44 (1H, s), 6.55 (1H, s), 6.91 (1H, d, J=9.0 Hz), 7.81 (1H, d, J=9.0 Hz).

### Example 8

In the same manner as that of Example 1, MIC values of M-246, M-21, J-Q21, and J-Q12.1 for Staphylococcus aureus were measured. M-246 and M-21 were obtained as commercial products, and as J-Q21 and J-Q12.1, those synthesized in Examples 6 and 7 were used. The MIC values (mg/L) for the quinolone-resistant methicillin-resistant Staphylococcus aureus (MRSA) Mu50 and quinolone-susceptible Staphylococcus aureus FDA209P are shown in Table 17.

TABLE 17

| Test compound | Mu50 | FDA209P |
|---|---|---|
| M-246 | 4 | 128 |
| M-21 | 16 | 64 |
| J-Q21 | 4 | >128 |
| J-Q12.1 | 8 | 8 |

### Example 9

The DNA cleavage assay was performed with M-246 in the same manner as that used in Example 4. The results are shown in Table 18.

TABLE 18

| | | GyrA(wt) + GyrB(wt) | | | GyrA(S84L) + GyrB(wt) | | |
|---|---|---|---|---|---|---|---|
| | Concentration | Ratio of substrate | Ratio of cleaved | IC₅₀ | Ratio of substrate | Ratio of cleaved | IC₅₀ |
| Compound | [μg/mL] | plasmid DNA (%) | DNA (%) | (μg/mL) | plasmid DNA (%) | DNA (%) | (μg/mL) |
| M246 | 0 | 92 | 8 | 16 | 93 | 7 | 1 |
| | 0.5 | 82 | 18 | | 54 | 46 | |
| | 1 | 77 | 23 | | 47 | 53 | |
| | 2 | 71 | 29 | | 39 | 61 | |
| | 4 | 60 | 40 | | 20 | 80 | |
| | 8 | 53 | 47 | | 16 | 84 | |
| | 16 | 44 | 56 | | 10 | 90 | |
| | 32 | 33 | 67 | | 5 | 95 | |
| Levofloxacin | 4 | 27 | 73 | | 83 | 17 | |

### INDUSTRIAL APPLICABILITY

The antibacterial agent of the present invention can exhibit potent antibacterial activity against bacteria that have acquired resistance to quinolones, especially a quinolone-resistant Staphylococcus aureus. Therefore, the agent is useful for a prophylactic and/or therapeutic treatment of, for example, MRSA infectious disease, VISA infectious disease, VRSA infectious disease, and the like. Further, when the antibacterial agent of the present invention is used in combination with a quinolone, the antibacterial agent of the present invention acts on bacteria that have acquired resistance to quinolones, and the quinolone acts on quinolone-susceptible bacteria. Therefore, an extremely effective prophylactic and/or therapeutic treatment can be achieved irrespective of the type of etiologic bacterium. Furthermore, if a quinolone and the antibacterial agent of the present invention are used in combination, emergence frequency of quinolone-resistant bacteria can be notably suppressed.

What is claimed is:

1. A prophylactic and/or therapeutic method of treatment for an infectious disease caused by a bacterium that has acquired resistance to quinolones, the method comprising administering as an active ingredient an antibacterial agent comprising a compound represented by the following general formula (I): 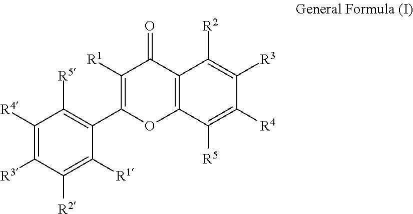
wherein, R¹ represents a hydrogen atom, a hydroxyl group, or a lower alkoxy group; R² represents a hydrogen atom or hydroxyl group; R³ represents a hydrogen atom or a hydroxyl group; R⁴ represents a hydrogen atom, a hydroxyl group, or a lower alkoxy group; R⁵ represents a hydrogen atom, a lower cyclic alkyl group, or a lower alkoxy group; R^{1′} represents a hydrogen atom; R^{2′} represents a hydrogen atom, hydroxyl group, or a lower alkoxy group; R^{3′} represents a hydrogen atom, a hydroxyl group, or a lower alkoxy group; R^{4′} represents a hydrogen atom or a hydroxyl group; and R^{5′} represents a hydrogen atom, to a subject in need thereof.

2. The method according to claim 1, wherein R¹ is a hydrogen atom, a hydroxyl group, or a methoxy group, R⁴ is a hydrogen atom, a hydroxyl group, or a methoxy group, R⁵ is a hydrogen atom, a cyclopropyl group, or a methoxy group, R^{2′} is a hydrogen atom, a hydroxyl group, or a methoxy group, and R^{3′} is a hydrogen atom, a hydroxyl group, or a methoxy group.

3. The method according to claim 1, wherein the bacterium has acquired resistance to quinolones through introduction of a mutation into gyrase.

4. The method according to claim 1, which comprises administering, as an active ingredient, a compound selected from the group consisting of:
a compound of the general formula (I), wherein R¹ is a hydroxyl group, R² is a hydroxyl group, R³ is a hydrogen atom, R⁴ is a hydroxyl group, R⁵ is a hydrogen atom, R^{1′} is a hydrogen atom, R^{2′} is a hydroxyl group, R^{3′} is a hydroxyl group, R^{4′} is a hydrogen atom, and R^{5′} is a hydrogen atom;
a compound of the general formula (I), wherein R¹ is a hydroxyl group, R² is a hydroxyl group, R³ is a hydrogen atom, R⁴ is a hydroxyl group, R⁵ is a hydrogen atom, R^{1′} is a hydrogen atom, R^{2′} is a hydrogen atom, R^{3′} is a hydroxyl group, R^{4′} is a hydrogen atom, and R^{5′} is a hydrogen atom;
a compound of the general formula (I), wherein R¹ is a hydroxyl group, R² is a hydroxyl group, R³ is a hydrogen atom, R⁴ is a hydroxyl group, R⁵ is a methoxy group, R^{1′} is a hydrogen atom, R^{2′} is a methoxy group, R^{3′} is a hydroxyl group, R^{4′} is a hydrogen atom, and R^{5′} is a hydrogen atom;
a compound of the general formula (I), wherein R¹ is a hydrogen atom, R² is a hydroxyl group, R³ is a hydrogen atom, R⁴ is a hydroxyl group, R⁵ is a hydrogen atom, R^{1′} is a hydrogen atom, R^{2′} is a hydroxyl group, R^{3′} is a hydroxyl group, R^{4′} is a hydrogen atom, and R^{5′} is a hydrogen atom;
a compound of the general formula (I), wherein R¹ is a hydrogen atom, R² is a hydroxyl group, R³ is a hydrogen atom, R⁴ is a hydroxyl group, R⁵ is a hydrogen atom, R^{1′} is a hydrogen atom, R^{2′} is a hydrogen atom, R^{3′} is a hydroxyl group, R^{4′} is a hydrogen atom, and R^{5′} is a hydrogen atom;
a compound of the general formula (I), wherein R¹ is a hydrogen atom, R² is a hydrogen atom, R³ is a hydrogen atom, R⁴ is a methoxy group, R⁵ is a hydrogen atom, R^{1′} is a hydrogen atom, R^{2′} is a hydrogen atom, R^{3′} is a hydrogen atom, R^{4′} is a hydrogen atom, and R^{5′} is a hydrogen atom;
a compound of the general formula (I), wherein R¹ is a hydrogen atom, R² is a hydrogen atom, R³ is a hydrogen atom, R⁴ is a methoxy group, R⁵ is a hydrogen atom, R^{1′} is a hydrogen atom, R^{2′} is a hydrogen atom, R^{3′} is a hydroxyl group, R^{4′} is a hydrogen atom, and R^{5′} is a hydrogen atom;
a compound of the general formula (I), wherein R¹ is a methoxy group, R² is a hydroxyl group, R³ is a hydrogen atom, R⁴ is a methoxy group, R⁵ is a hydrogen atom, R^{1′} is a hydrogen atom, R^{2′} is a methoxy group, R^{3′} is a methoxy group, R^{4′} is a hydroxyl group, and R^{5′} is a hydrogen atom;
a compound of the general formula (I), wherein R¹ is a methoxy group, R² is a hydroxyl group, R³ is a hydrogen atom, R⁴ is a methoxy group, R⁵ is a hydrogen atom, R^{1′} is a hydrogen atom, R^{2′} is a methoxy group, R^{3′} is a hydroxyl group, R^{4′} is a hydrogen atom, and R^{5′} is a hydrogen atom;
a compound of the general formula (I), wherein R¹ is a hydrogen atom, R² is a hydrogen atom, R³ is a hydrogen atom, R⁴ is a methoxy group, R⁵ is a hydrogen atom, R^{1′} is a hydrogen atom, R^{2′} is a methoxy group, R^{3′} is a hydroxyl group, R^{4′} is a hydrogen atom, and R^{5′} is a hydrogen atom;
a compound of the general formula (I), wherein R¹ is a hydroxyl group, R² is a hydroxyl group, R³ is a hydroxyl group, R⁴ is a hydroxyl group, R⁵ is a hydrogen atom, R^{1′} is a hydrogen atom, R^{2′} is a hydrogen atom, R^{3′} is a hydrogen atom, R^{4′} is a hydrogen atom, and R^{5′} is a hydrogen atom;
a compound of the general formula (I), wherein R¹ is a hydrogen atom, R² is a hydrogen atom, R³ is a hydrogen atom, R⁴ is a hydroxyl group, R⁵ is a cyclopropyl group, R^{1′} is a hydrogen atom, R^{2′} is a hydrogen atom, R^{3′} is a hydroxyl group, R^{4′} is a hydrogen atom, and R^{5′} is a hydrogen atom; and
a compound of the general formula (I), wherein R¹ is a hydrogen atom, R² is a hydroxyl group, R³ is a hydrogen atom, R⁴ is a hydroxyl group, R⁵ is a cyclopropyl group, R^{1′} is a hydrogen atom, R^{2′} is a hydrogen atom, R^{3′} is a hydroxyl group, R^{4′} is a hydrogen atom, and R^{5′} is a hydrogen atom.

5. The method according to claim 1, wherein the bacterium is Staphylococcus aureus.

6. A method of suppressing emergence and/or proliferation of a quinolone-resistant bacterium, comprising contacting a quinolone-resistant bacterium with a compound represented by the general formula (I) according to claim 1 and a quinolone antibacterial agent.

7. The method according to claim 6, wherein the bacterium that has acquired resistance to quinolones through introduction of a mutation into gyrase.

8. The method according to claim 6, further comprising therapeutic treatment of an infectious disease.

9. The method according to claim 1, which comprises administering a combination of the antibacterial agent according to claim 1 and a quinolone.

10. The method according to claim 1, which comprises administering an antibacterial agent comprising apigenin, myricetin 3,7,3′,4′-tetramethyl ether, isopratol, luteolin, or kaempferol hydrate.

11. A method of inhibiting a DNA gyrase of a bacterium that has acquired resistance to a quinolone comprising:
contacting the bacterium with a compound represented by the general formula (I) according to claim 1 as an active ingredient; and
inhibiting a DNA gyrase of the bacterium.

12. The method according to claim 11, which suppresses emergence and/or proliferation of a quinolone-resistant bacterium.

13. The method according to claim 2, which comprises administering, as an active ingredient, a compound selected from the group consisting of:
a compound of the general formula (I), wherein R¹ is a hydroxyl group, R² is a hydroxyl group, R³ is a hydrogen atom, R⁴ is a hydroxyl group, R⁵ is a hydrogen atom, R^{1′} is a hydrogen atom, R^{2′} is a hydroxyl group, R^{3′} is a hydroxyl group, R^{4′} is a hydrogen atom, and R^{5′} is a hydrogen atom;
a compound of the general formula (I), wherein R¹ is a hydroxyl group, R² is a hydroxyl group, R³ is a hydrogen atom, R⁴ is a hydroxyl group, R⁵ is a hydrogen atom, R^{1′} is a hydrogen atom, R^{2′} is a hydrogen atom, R^{3′} is a hydroxyl group, R^{4′} is a hydrogen atom, and R^{5′} is a hydrogen atom;
a compound of the general formula (I), wherein R¹ is a hydroxyl group, R² is a hydroxyl group, R³ is a hydrogen atom, R⁴ is a hydroxyl group, R⁵ is a methoxy group, R^{1′} is a hydrogen atom, R^{2′} is a methoxy group, R^{3′} is a hydroxyl group, R^{4′} is a hydrogen atom, and R^{5′} is a hydrogen atom;
a compound of the general formula (I), wherein R¹ is a hydrogen atom, R² is a hydroxyl group, R³ is a hydrogen atom, R⁴ is a hydroxyl group, R⁵ is a hydrogen atom, R^{1′} is a hydrogen atom, R^{2′} is a hydroxyl group, R^{3′} is a hydroxyl group, R^{4′} is a hydrogen atom, and R^{5′} is a hydrogen atom;
a compound of the general formula (I), wherein R¹ is a hydrogen atom, R² is a hydroxyl group, R³ is a hydrogen atom, R⁴ is a hydroxyl group, R⁵ is a hydrogen atom, R^{1′} is a hydrogen atom, R^{2′} is a hydrogen atom, R^{3′} is a hydroxyl group, R^{4′} is a hydrogen atom, and R^{5′} is a hydrogen atom;
a compound of the general formula (I), wherein R¹ is a hydrogen atom, R² is a hydrogen atom, R³ is a hydrogen atom, R⁴ is a methoxy group, R⁵ is a hydrogen atom, R^{1′} is a hydrogen atom, R^{2′} is a hydrogen atom, R^{3′} is a hydrogen atom, R^{4′} is a hydrogen atom, and R^{5′} is a hydrogen atom;
a compound of the general formula (I), wherein R¹ is a hydrogen atom, R² is a hydrogen atom, R³ is a hydrogen atom, R⁴ is a methoxy group, R⁵ is a hydrogen atom, R^{1′} is a hydrogen atom, R^{2′} is a hydrogen atom, R^{3′} is a hydroxyl group, R^{4′} is a hydrogen atom, and R^{5′} is a hydrogen atom;
a compound of the general formula (I), wherein R¹ is a methoxy group, R² is a hydroxyl group, R³ is a hydrogen atom, R⁴ is a methoxy group, R⁵ is a hydrogen atom, R^{1′} is a hydrogen atom, R^{2′} is a methoxy group, R^{3′} is a methoxy group, R^{4′} is a hydroxyl group, and R^{5′} is a hydrogen atom;
a compound of the general formula (I), wherein R¹ is a methoxy group, R² is a hydroxyl group, R³ is a hydrogen atom, R⁴ is a methoxy group, R⁵ is a hydrogen atom, R^{1′} is a hydrogen atom, R^{2′} is a methoxy group, R^{3′} is a hydroxyl group, R^{4′} is a hydrogen atom, and R^{5′} is a hydrogen atom;
a compound of the general formula (I), wherein R¹ is a hydrogen atom, R² is a hydrogen atom, R³ is a hydrogen atom, R⁴ is a methoxy group, R⁵ is a hydrogen atom, R^{1′} is a hydrogen atom, R^{2′} is a methoxy group, R^{3′} is a hydroxyl group, R^{4′} is a hydrogen atom, and R^{5′} is a hydrogen atom;
a compound of the general formula (I), wherein R¹ is a hydroxyl group, R² is a hydroxyl group, R³ is a hydroxyl group, R⁴ is a hydroxyl group, R⁵ is a hydrogen atom, R^{1′} is a hydrogen atom, R^{2′} is a hydrogen atom, R^{3′} is a hydrogen atom, R^{4′} is a hydrogen atom, and R^{5′} is a hydrogen atom;
a compound of the general formula (I), wherein R¹ is a hydrogen atom, R² is a hydrogen atom, R³ is a hydrogen atom, R⁴ is a hydroxyl group, R⁵ is a cyclopropyl group, R^{1′} is a hydrogen atom, R^{2′} is a hydrogen atom, R^{3′} is a hydroxyl group, R^{4′} is a hydrogen atom, and R^{5′} is a hydrogen atom; and
a compound of the general formula (I), wherein R¹ is a hydrogen atom, R² is a hydroxyl group, R³ is a hydrogen atom, R⁴ is a hydroxyl group, R⁵ is a cyclopropyl group, R^{1′} is a hydrogen atom, R^{2′} is a hydrogen atom, R^{3′} is a hydroxyl group, R^{4′} is a hydrogen atom, and R^{5′} is a hydrogen atom.
